(19) United States
(12) Patent Application Publication
(10) Pub. No.: US 2009/0099177 A1
(43) Pub. Date: Apr. 16, 2009

(54) PYRIDYL COMPOUNDS

(76) Inventors: Gerard Martin Paul Giblin, Essex (GB); Adrian Hall, Essex (GB); David Nigel Hurst, Essex (GB); Tiziana Scoccitti, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/299,120

(22) PCT Filed: May 2, 2007
(86) PCT No.: PCT/EP2007/054254

§ 371 Date: Oct. 31, 2008

(30) Foreign Application Priority Data
May 4, 2006 (GB) 0608825.6

(51) Int. Cl. A61K 31/5377; C07D 213/74; A61K 31/44; C07D 413/14; C07D 473/00; A61K 31/52

(52) U.S. Cl. 514/234.5; 546/309; 514/352; 544/277; 514/263.22; 544/124

(57) ABSTRACT

Compounds of formula (I) or a pharmaceutically acceptable derivative thereof: wherein R¹, and R², and R³ are as defined in the specification, a process for the preparation of such compounds, pharmaceutical compositions comprising such compounds and the use of such compounds in medicine.

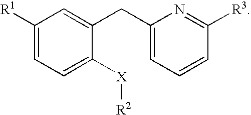

This invention relates to pyridyl compounds, to processes for their preparation, to pharmaceutical compositions containing them and to their use in medicine, in particular their use in the treatment of conditions mediated by the action of PGE₂ at the EP₁ receptor.

Prostaglandin receptors, including the EP₁₋₄, DP, FP IP and TP receptors are the effector proteins for the products (prostaglandins) downstream of COX-1/2 activation (PGE₂, PGD2, PGF2a, PGl2 and thromboxane respectively). The NSAIDS (non-steroidal anti-inflammatory drugs) are indiscriminate cyclooxygenase inhibitors and reduce the levels of these prostaglandins. This in turn reduces the action of the prostaglandins at their respective receptors. In view of the relatively large number of receptors affected, the pharmacology of the NSAIDS is complex.

The EP₁ receptor is a 7-transmembrane receptor and its natural ligand is the prostaglandin PGE₂, PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion.

We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006) and WO 2006/114313 (2 Nov. 2006) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al., Biorg. Med. Chem. Lett., 2005, 15(4); 1155 also discloses 2,3-diarylthiophenes as selective EP₁ receptor antagonists.

S. C. McKeown et al, Bioorg. Med. Chem. Lett., 2007, 17, 1750; A. Hall et al, Bioorg. Med. Chem. Lett., 2007, 17, 1200; A. Hall et al, Bioorg. Med. Chem. Lett., 2007, 17, 916; A. Hall et al., Bioorg. Med. Chem. Lett., 2007, 17, 732; G. M. P. Giblin et al., Bioorg. Med. Chem. Lett., 2007, 17, 385-389; S. C. McKeown et al., Bioorg. Med. Chem. Lett., 2006, 16 (18), 4767-4771; “A. Hall et al, Bioorg. Med. Chem. Lett., 2006, 16 (14), 3657-3662; and A. Hall et al., Bioorg. Med. Chem. Lett., 2006, 16 (10), 2666-2671 relate to EP₁ receptor antagonist compounds.

It is now suggested that a novel group of pyridine derivatives are indicated to be useful in treating conditions mediated by the action of PGE₂ at EP₁ receptors. Such conditions include pain, or inflammatory, immunological, bone, neurodegenerative or renal disorders.

Accordingly the present invention provides one or more chemical entities selected from compounds of formula (I):

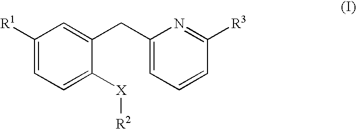

wherein:
R¹ represents halogen;
X represents oxygen or sulfur;
R² represents isobutyl or optionally substituted benzyl;
R³ represents —CO—NH—(CH₂)ₘ—R⁴, —NH—COO—R⁵, —NH—CO—(CH₂)ₙ—R⁶, —C(H)(OH)—CF₃, or R³ represents optionally substituted imidazolyl wherein optionally the imidazole ring is fused to give an optionally substituted bicyclic or tricyclic ring system;
R⁴ represents hydrogen, C₃₋₈ alkyl, C₃₋₈ cycloalkyl, optionally substituted phenyl or optionally substituted pyridyl;
R⁵ represents t-butyl;
R⁶ represents C₃₋₈ alkyl, C₃₋₈ cycloalkyl, optionally substituted phenyl, optionally substituted pyridyl, tetrahydropyranyl or tetrahydrofuranyl;
m and n independently represents 0 or 1;
and derivatives thereof.

Optional substituents for phenyl, benzyl or pyridyl moieties are selected from optionally substituted C₁₋₆alkyl, optionally substituted C₁₋₆alkoxy, halogen, HOC₁₋₄alkyl (e.g. HOCH₂), amino (e.g. NMe₂, —CH₂—NHMe, —CH₂—NMe₂ or —CH₂—N(Me)(cyanoethyl)), CH₂heterocyclyl (e.g. CH₂pyrrolidine, CH₂piperidine or CH₂morpholine), C₁₋₄alkylheterocyclyl-CH₂— (e.g. 4-methylpiperazine-CH₂—).

Suitably, R¹ is chlorine.

Suitably, X represents oxygen.

Suitably, R² is isobutyl or benzyl optionally substituted by one or more halogen atoms (e.g. fluorine and chlorine; such as 2-fluoro-4-chlorobenzyl).

Suitably, R³ is —CO—NH—(CH₂)ₘ—R⁴ (e.g. —CO—NH-pyridyl, —CO—NH—CH₂-pyridyl, —CO—NH-t-butyl, —CO—NH-isopropyl, —CO—NH-phenyl, —CO—NH—CH₂-phenyl, —CO—NH-cyclohexyl or —CONH₂).

Suitably, R⁴ represents hydrogen, C₃₋₈ alkyl (e.g. t-butyl, isopropyl), C₃₋₈ cycloalkyl (e.g. cyclohexyl), optionally substituted phenyl or optionally substituted pyridyl;

When R⁴ represents optionally substituted phenyl or optionally substituted pyridyl, suitable optional substituents are selected from HOC₁₋₄alkyl (e.g. HOCH₂), amino (e.g. —CH₂—NMe₂), —CH₂—N(Me)(cyanoethyl) or CH₂heterocyclyl (e.g. CH₂pyrrolidine, CH₂piperidine or CH₂morpholine).

Suitably, R³ is —NH—COO—R⁵ (e.g. —NH—COO-t-butyl).

Suitably, R³ is —NH—CO—(CH₂)ₙ—R⁶ (e.g. —NH—CO-phenyl, —NH—CO—CH₂-phenyl, —NH—CO-cyclohexyl, —NH—CO—CH₂-t-butyl, —NH—CO-pyridyl, —NH—CO-tetrahydropyranyl or —NH—CO-tetrahydrofuranyl).

Suitably, R⁶ represents C₃₋₈ alkyl (e.g. t-butyl), C₃₋₈ cycloalkyl (e.g. cyclohexyl), optionally substituted phenyl, optionally substituted pyridyl, tetrahydropyranyl or tetrahydrofuranyl;

When R⁶ represents optionally substituted phenyl or optionally substituted pyridyl, suitably optional substituents are selected from HOC₁₋₄alkyl (e.g. HOCH₂), CH₂heterocyclyl (e.g. CH₂pyrrolidine, CH₂piperidine or CH₂morpholine) or C₁₋₄alkylheterocyclyl-CH₂— (e.g. 4-methylpiperazine-CH₂—).

Suitably, R³ is —C(H)(OH)—CF₃.

Examples of fused imidazole groups of R³ include benzimidazole, imidazo[1,2-a]pyridine, imidazo[1,2-a]pyrazine, imidazo[1,2-a]pyrimidine, imidazo[4,5-b]pyridine, imidazo[4,5-b]pyrazine, imidazo[4,5-c]pyridine, purine, imidazo[4,5-c]quinoline, dihydroimidazo[4,5-e][1,2,3]benzotriazole and dihydroimidazo[4,5-f]indazole all of which may be optionally substituted. Suitable optional substituents include one or two substituents selected from halogen (e.g. Cl or F); alkyl (e.g. methyl), alkylamino (e.g. NMe₂, —CH₂—NHMe or —CH₂—NMe₂), heterocyclyl (e.g. morpholinyl), C₁₋₄alkylheterocyclyl (e.g. 4-methylpiperidinyl, or 4-methylpiperazine); OC₁₋₄alkyl, (e.g. OCH₃); HOC₁₋₄alkyl (e.g. HOCH₂); CH₂NHC₁₋₄alkyl; CH₂N(C₁₋₄alkyl)₂ or CH₂heterocyclyl (e.g. CH₂pyrrolidine, CH₂piperidine or CH₂morpholine).

Suitably, R³ is imidazole, benzimidazole, purine, imidazo[4,5-b]pyridine, imidazo[4,5-c]pyridine, imidazo[4,5-b]pyrazine, dihydroimidazo[4,5-f]indazole, imidazo[4,5-c]quinoline or dihydroimidazo[4,5-e][1,2,3]benzotriazole, each of which may be optionally substituted by one or two substituents selected from halogen (e.g. Cl or F), alkyl (e.g. methyl), amino (e.g. —CH₂—NHMe or —CH₂—NMe₂), heterocyclyl (e.g. morpholinyl), C₁₋₄alkylheterocyclyl (e.g. 4-methylpiperazine), OC₁₋₄alkyl, (e.g. OCH₃) or CH₂heterocyclyl (e.g. CH₂pyrrolidine, or CH₂piperidine).

Compounds of formula (I) include the compounds of Examples 1 to 63 and derivatives thereof.

Particular Examples of compounds of Formula (I) include the compounds of Examples 17, 18, 19, 20, 28, 30, 33, 43, 44 and 51.

Certain compounds of the examples are selective for EP₁ over EP₃. Certain compounds of the Examples have greater than 10 fold selectivity. Certain compounds of the Examples have greater than 30 fold selectivity.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

Compounds of formula (I) can be prepared as set forth in the following schemes and in the examples. The following processes form another aspect of the present invention.

For example, compounds of formula (II) may be prepared by the general route shown in Scheme I below:

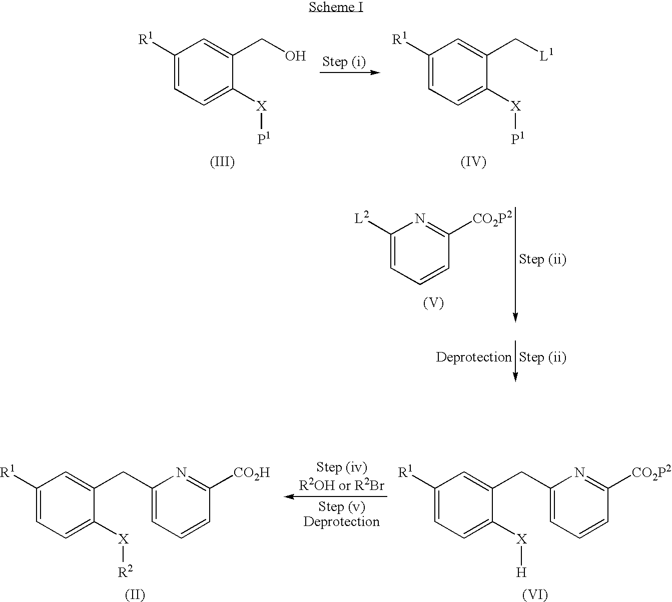

wherein R¹, X and R² are as defined for compounds of formula (I), L¹ and L² are suitable leaving groups (such as a halo group selected for example from bromo and iodo) and P¹ and P² are suitable protecting groups known to the skilled person, for example, P¹ and P² are suitably C₁₋₄alkyl or optionally substituted benzyl (e.g. P¹ is suitably benzyl when X represents oxygen).

Suitable conditions for step (i) include treating a compound of formula (III) with phosphorous tribromide in a suitable solvent such as dichloromethane.

Suitable conditions for step (ii) comprises reaction of a compound of formula (IV) with a compound of formula (V) to give a compound of formula (VI) include treating the compound of formula (IV) with activated zinc in a suitable solvent, e.g. tetrahydrofuran, and adding the resulting reagent to the compound of formula (V) in the presence of tetrakis(triphenylphosphine)palladium(0).

Removal of the protecting group P¹ in step (iii) can be achieved by heating with sodium methanethiolate in N,N-dimethylformamide. The skilled person will recognise that this procedure may also result in the loss of the P² group. A protecting group may be replaced by conventional means.

Step (iv) may typically be performed by reacting a compound of formula (VI) with a suitable source of R² wherein R² is as defined for a compound of formula (I). Suitable sources of R² include but are not limited to R²OH, R²Br, R²OTs and R²OMs. Suitable reaction conditions when the source of R² is R²Br includes heating in the presence of a base e.g. potassium carbonate in a suitable solvent e.g. acetone or N,N-dimethylformamide. Alternatively step (iv) may be prepared by the reaction with R²OH under Mitsunobu conditions (Ph₃P/diisopropylazodicarboxylate) (O. Mitsunobu et al., Bull. Chem. Soc. Japan, 40, 935 (1967); O. Mitsunobu, Y. Yamada, ibid. 2380).

Step (v) typically comprises removal of protecting group P² by suitable deprotection methods known to the skilled person. Conditions for the deprotection of an ester to give the corresponding carboxylic acid are known to those skilled in the art and include heating in the presence of a suitable base, e.g. aqueous sodium hydroxide, in a solvent e.g. an alcohol.

Alternatively compounds of formula (II) may also be prepared by the general route shown in Scheme II:

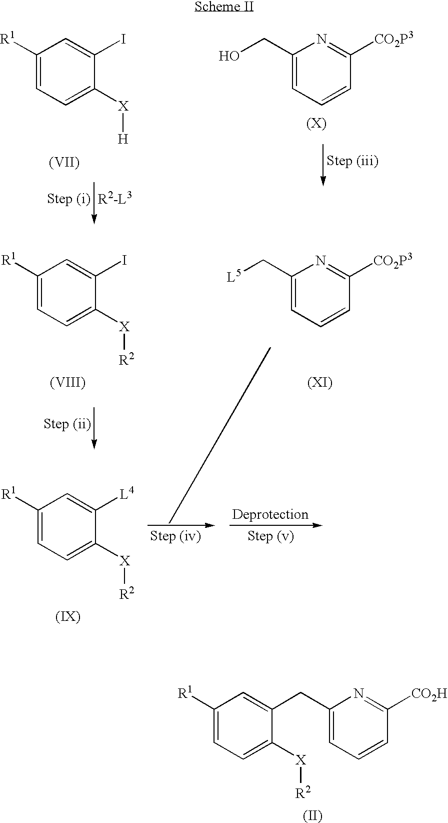

wherein R¹, X and R² are as defined for compounds of formula (I), P³ is a suitable protecting group (e.g. methyl or ethyl), L³ is a leaving group (e.g. Br), L⁴ is an activating group e.g. boronic acid or a boronic ester and L⁵ is a leaving group (e.g. Cl).

Step (i) may be performed by reaction of a compound of formula (VII) with R²L³. Suitable reaction conditions include heating the compounds together in the presence of a base (e.g. potassium carbonate) in a suitable solvent, for example acetone.

When L⁴ represents B(OH)₂, step (ii) may be performed according to conventional methods from the corresponding iodobenzene of formula (VIII) by treatment with iso-propylmagnesium bromide followed by trimethyl borate in a suitable solvent such as tetrahydrofuran under anhydrous conditions in an inert atmosphere, followed by treatment with aqueous hydrochloric acid. When L⁴ represents a boronic ester, step (ii) may be prepared under similar conditions, and by using, for example, isopropyltetramethyldioxaborolane instead of trimethyl borate.

When L⁵ is chloro, step (iii) may be performed by reacting the compound of formula (X) with thionyl chloride in a suitable solvent such as dichloromethane.

Step (iv) may typically be performed by reaction of a compound of formula (IX) with a compound of formula (XI). Suitably the compound of formula (IX) is a boronic acid [L⁴ is B(OH)₂] or a boronic ester [L⁴ is e.g. 4,4,5,5,-tetramethyl-1,3,2-dioxaborolane].

When the compound of formula (IX) is a boronic acid or ester and L⁵ represents chloro, step (iv) typically comprises heating the intermediates in the presence of tetrakis(triphenylphosphine)palladium(0) and a base, e.g. potassium carbonate, in a suitable solvent system (e.g. from 1:1 to 15:1 toluene/ethanol).

Step (v) typically comprises removal of protecting group P³ by suitable deprotection methods known to the skilled person. Conditions for the deprotection of an ester to give the corresponding carboxylic acid are known to those skilled in the art and include heating in the presence of a suitable base, e.g. aqueous sodium hydroxide, in a solvent e.g. an alcohol.

It will be recognised to those skilled in the art that the compounds of formula (I) can be derived from the corresponding carboxylic acid derivative of formula (II). For example, compounds wherein R³ is an amide (e.g. —CO—NH—(CH₂)ₘ—R⁴), can be prepared by activation of the carboxylic acid, for example by forming the acid chloride (for example by reaction of the carboxylic acid with thionyl chloride) or by activation with EDAC (N-(3-dimethylaminopropyl)-N′-ethylcarbodiimide hydrochloride) in the presence of HOBt (1-hydroxybenzotriazole) (as detailed in the examples) followed by reaction with an amine respectively. Other derivatives, for example when R³ is NHCO₂R⁵ may be accessed by using the Curtius reaction (P. A. S. Smith, Org. React. 3, 337-449 (1946) and J. H. Saunders, R. J. Slocombe, Chem. Rev. 43, 205 (1948)), with a suitable alcohol. Derivatives where R³ is NHCO(CH₂)ₙR⁶ may also be prepared via the aforementioned Curtius reaction with a suitable alcohol followed by deprotection of the resulting carbamate and reaction with a carboxylic acid derivative such as an acid chloride.

Compounds of formula (I) wherein R³ is an imidazole moiety fused to give an optionally substituted bicyclic or tricyclic ring system [hereinafter referred to as compounds of formula (I)^{a}] may be prepared from compounds of formula (XII) following the methods described in, for example, A. Czarny et al, J. Het. Chem., 1996, 33(4), 1393-1398 and according to the following Scheme III:

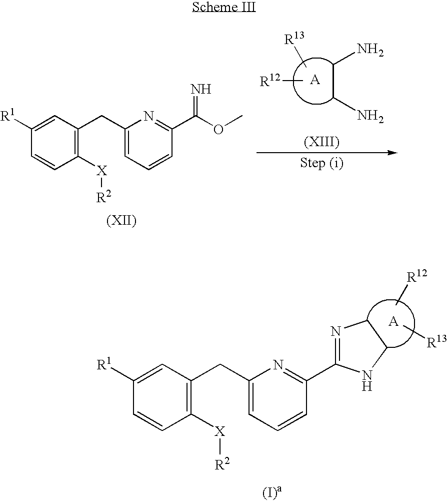

wherein R¹, R² and X are as defined for compounds of formula (I), A represents e.g. phenyl, pyridine, quinoline, or thiophene, and R¹² and R¹³ each represent hydrogen or a substituent.

Step (i) may typically be performed by heating the intermediates together in a suitable solvent e.g. ethanol.

Compounds of formula (XII) may be prepared from the corresponding carboxylic acid of formula (II) by known methods, for example as described in the examples. Suitable methods include the reaction of a compound of formula (II) with thionyl chloride then ammonia, then phosphorus oxychloride, then sodium methoxide in methanol.

Compounds of formula (I) wherein R³ is an imidazole may be prepared by reaction of a compound of formula (XII) with a suitable reagent, such as 2,2-bis(methyloxy)ethanamine (aminoacetaldehyde dimethyl acetal) as described in the examples.

Compounds of formula (III), (V), (VII), (X) and (XIII) are either commercially available, or may be prepared by known methods.

Accordingly the present invention also provides a process for the preparation of a compound of formula (I) or a derivative thereof:

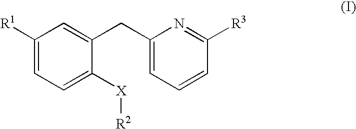

wherein:
R¹ represents halogen;
X represents oxygen or sulfur;
R² represents isobutyl or optionally substituted benzyl;
R³ represents —CO—NH—(CH₂)ₘ—R⁴, —NH—COO—R⁵, —NH—CO—(CH₂)ₙ—R⁶, —C(H)(OH)—CF₃, or R³ represents optionally substituted imidazolyl wherein optionally the imidazole ring is fused to give an optionally substituted bicyclic or tricyclic ring system;
R⁴ represents hydrogen, C₃₋₈ alkyl, C₃₋₆ cycloalkyl, optionally substituted phenyl or optionally substituted pyridyl;
R⁵ represents t-butyl;
R⁶ represents C₃₋₈ alkyl, C₃₋₈ cycloalkyl, optionally substituted phenyl, optionally substituted pyridyl, tetrahydropyranyl or tetrahydrofuranyl;
m and n independently represents 0 or 1;
or derivatives thereof;
comprising:
converting a compound of formula (II):

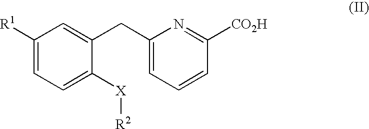

wherein R¹, R² and X are as defined for compounds of formula (I);
to a compound of formula (I);
and if required, and in any order;
converting one group R³ to another group R³; and/or
effecting deprotection; and/or
forming a derivative thereof.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. Nos. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt.

### Abbreviations

AcOH (acetic acid), Bn (benzyl), Boc (tert-butoxycarbonyl), Bu, Pr, iPr, Me, Et (butyl, propyl, isopropyl, methyl, ethyl), DBU (1,8-diazabicyclo[5.4.0]undec-7-ene), DMSO (dimethyl sulfoxide), DCMIMDC (dichloromethane), DME (ethylene glycol dimethyl ether), DMF (N,N-dimethylformamide), DMP (Dess-Martin periodinane), DPPA (diphenyl phosphoryl azide), EDAC/EDC (N-(3-dimethylaminopropyl)-N′-ethylcarbodiimide), EDTA (ethylenediaminetetraacetic acid), EtOAc (ethyl acetate), EtOH (ethanol), Et₂O (diethyl ether), HOBT/HOBt (1-hydroxybenzotriazole), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NMR (Nuclear Magnetic Resonance (spectrum)), NMP (n-methylpyrrolidone), Ph (phenyl), PhCH₃ (toluene), i-PrOH (isopropanol), pTSA (para-toluene sulfonic acid), ppt (precipitate), RT/Rt (retention time), SM (starting material), SPE (Solid Phase Extraction-silica cartridge chromatography), TBAF (tetrabutylammonium fluoride), TBME (tertiary butyl methyl ether), TEA (triethylamine), TFA (trifluoroacetic acid), TFAA (trifluoroacetic anhydride), THF (tetrahydrofuran), s, d, dd, t, q, m, br (singlet, doublet, double doublet, triplet, quartet, multiplet, broad.)

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al., Int. J. Mass Spectrom., 2004, 238, 153-162.

The terms “Biotage®”, “Biotage 75” and “Biotage SP4®” when used herein refer to commercially available automated purification systems using pre-packed silica gel cartridges. The term FLEX (or Parallel Flex) when used herein refers to a parallel HPLC purification system.

### LCMS

The following LCMS conditions were used during the preparation of the examples.

### Software

Waters MassLynx version 4.0 SP2

### Column

The column used is a Waters Atlantis, the dimensions of which are 4.6 mm×50 mm. The stationary phase particle size is 3m.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Preparations

### Description 1

### Sodium 6-[(5-chloro-2-{[(4-chloro-2-fluorophenyl)methyl]oxy}phenyl)methyl]-2-pyridinecarboxylate (D1)

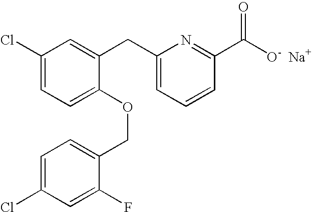

### Step (a) 4-Chloro-2-iodophenol

Boron tribromide (1349 g) was added to a solution of 4-chloro-2-iodoanisole (1025 g) in dichloromethane (10.3 L) under nitrogen at such a rate that the temperature remained at 0-5° C. The solution was then warmed to 20° C. and stirred for c. 19 h until the reaction was complete by HPLC. This organic solution was added to water (8.2 L) and the mixture was cooled to 5° C. to 10° C. DCM (770 ml) was added and the resulting biphasic mixture was then stirred at 5° C. for 15 min before being warmed to 22° C. and then finally stirred at 22° C. for 20 min before separating the phases. The separated organic phase was washed with aqueous saturated sodium bicarbonate (3.1 L), water (3.1 L) and then evaporated on a Buchi to give the title compound. (963.6 g)

### Step (b) Ethyl 6-(chloromethyl)-2-pyridinecarboxylate

Thionyl chloride (13.8 ml) was added over ˜15 minutes to a stirred solution of ethyl 6-(hydroxymethyl)-2-pyridinecarboxylate (28.5 g) in MDC (200 ml) maintaining the temperature at 10-15° C. using an ice-water bath. On completion of the addition the mixture was stirred at room temperature for 1 hour. The solvent was evaporated and the residue partitioned between toluene (200 ml)/saturated bicarb (sodium bicarbonate solution, 200 ml). The layers were separated and the organic phase washed with water (150 ml). The solvent was evaporated to leave a pale oil which solidified on standing. (31.3 g).

### Step (c) 4-Chloro-1-{[(4-chloro-2-fluorophenyl)methyl]oxy}-2-iodobenzene

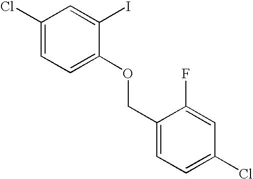

To a solution of 4-chloro-2-iodophenol (899 g, 1 eq) and 4-chloro-2-fluorobenzyl bromide (700 g, 1.02 eq) in acetone (8.1 L) was added anhydrous potassium carbonate (926 g). The stirred suspension was then heated to reflux for 30 minutes. 0.12% starting material was observed by HPLC. The product mixture was cooled to 20-25° C. HPLC showed complete consumption of starting material. Inorganic material was then removed by filtration. The residue was washed with acetone (3.6 L) and the combined filtrate and washes were concentrated to 5 vol by atmospheric distillation. Isooctane (4.5 L) was added and reconcentrated to 5 vol by atmospheric distillation. This was repeated once more. The solution was then cooled from 85° C. to 75° C. No precipitation occurred. The batch was then cooled further to 55° C. over 30 minutes, leading to the formation of an immobile suspension. The batch was re-heated to 65° C. which thinned the suspension. The batch was then cooled to 55° C. over 30 minutes. This caused a more controlled precipitation with a mobile suspension.

The batch was then cooled to 20° C. over 30 min. This led to a skin of product forming on all surfaces of the vessel whilst the suspension stayed mobile. The mixture was then stirred overnight at 20° C. The mixture was then cooled to −5° C. over 30 minutes and aged at −5° C. for 1.5 h. A crust formed on the bottom of the vessel. The mother liquors were recycled 4 times to remove this material. When the crust was dislodged, this wedged against the stirrer causing it to break at the top of the guide. The final recycle of mother liquors removed this from the vessel, following manual breaking with a long spatula. The solid was then collected by filtration. The filter cake was washed with iso-octane (1.5 L) chilled to −5° C. The solid was then dried in vacuo at 45° C. to a constant weight. Yield 1312.4 g.

### Step (d) 2-(5-chloro-2-{[(4-chloro-2-fluorophenyl)methyl]oxy}phenyl)-4,4,5,5-tetramethyl-1,3,2-dioxaborolane

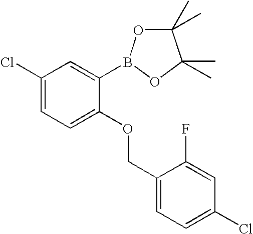

### Reaction 1

4-Chloro-1-{[(4-chloro-2-fluorophenyl)methyl]oxy}-2-iodobenzene (18.8 g) was dissolved in dry THF (188 ml) under N₂ and the solution cooled to −10° C. in a cardice (dry ice)/acetone bath. To the cooled solution was added isopropyl magnesium chloride (47 ml of 2M solution in diethyl ether) dropwise over 23 minutes maintaining the reaction temperature at −10° C. (max temp over addition −9° C. Min temp over addition −12° C.). After the addition was completed the residual chloride (isopropyl magnesium chloride) was washed into the reaction with dry THF (5 ml). The reaction mixture was stirred at −10° C. for 15 minutes then isopropyl tetramethyl dioxaborolane (23 ml) was added in one portion. Reaction exotherm (−10° C. to 5° C.). The cooling bath was removed and the reaction mixture allowed to warm to ambient temperature. The reaction was stirred at ambient temperature overnight under static N₂ flow.

The cloudy reaction mixture was quenched by the addition of 50% saturated ammonium chloride solution (188 ml) and the mixture stirred then separated. The aqueous phase was re-extracted with THF (50 ml). The bulked organic phases were washed with water (190 ml). Emulsion formed. Solid NaCl added to break emulsion, required heating with airgun to finish separation. The THF solution (still slightly cloudy) was evaporated under reduced pressure at 40° C. to leave a wet solid. Isopropyl alcohol (50 ml) was added and re-stripped to leave a white solid. Isopropyl alcohol (20 ml) was added and the white slurry cooled in an ice-bath for 30 minutes. Solid was filtered, washed with the mother liquor, then washed on the pad with IPA (10 ml, cold) and sucked dry on the pad. The solid was transferred to a dish and dried in a vacuum oven at 50° C. over weekend to give the title product (16.77 g). NMR showed clean product.

### Reaction 2

A solution of 4-chloro-1-{[(4-chloro-2-fluorophenyl)methyl]oxy}-2-iodobenzene (20 g, 50 mmol) in dry THF (200 ml) was cooled to −10° C. Isopropyl magnesium chloride (2M in THF, 50 ml, 100 mmol) was added dropwise over ˜15 mins, then the mixture was stirred at −10° C. for 15 mins. 2-Isopropoxy-4,4,5,5-tetramethyl-1,3,3-borolane (24.4 ml, 120 mmol) was added and the mixture was allowed to warm to room temperature and stirred for 18 h. TMBE (200 ml) and saturated NH₄Cl (200 ml) were added, and the layers separated. The organic phase was dried over MgSO₄ and evaporated to a white semi-solid. Trituration with isohexane (50 ml) gave a white solid. The solid was filtered off, washed with isohexane (20 ml) and dried in a vacuum oven at 50° C. for 18 h to give the title compound (16.2 g).

### Reaction 3

A solution of 4-chloro-1-{[(4-chloro-2-fluorophenyl)methyl]oxy}-2-iodobenzene (20 g, 50 mmol) in dry THF (200 ml) was cooled to −10° C. Isopropyl magnesium chloride (2M in diethyl ether, 50 ml, 100 mmol) was added dropwise over ˜15 mins, then the mixture was stirred at −10° C. for 15 mins. 2-Isopropoxy-4,4,5,5-tetramethyl-1,3,3-borolane (24.4 ml, 120 mmol) was added and the mixture was allowed to warm to room temperature and stirred for 18 h. TBME (200 ml) and saturated NH₄Cl (200 ml) were added, and the layers separated. The organic phase was washed with water (200 ml), dried over MgSO₄ and evaporated to a white semi-solid. Trituration with isohexane (50 ml) gave a white solid which was filtered, washed with isohexane (20 ml) and dried in a vacuum oven at 50° C. for 18 h to give the title compound (16.4 g).

### Step (e) Ethyl 6-[(5-chloro-2-{[(4-chloro-2-fluorophenyl)methyl]oxy}phenyl)methyl]-2-pyridinecarboxylate

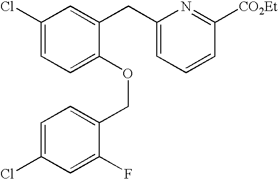

### Reaction 1

A mixture of 2-(5-chloro-2-{[(4-chloro-2-fluorophenyl)methyl]oxy}phenyl)-4,4,5,5-tetramethyl-1,3,2-dioxaborolane (8 g), ethyl 6-(chloromethyl)-2-pyridinecarboxylate (4 g), K₂CO₃ (5.6 g) and (tetrakis(triphenylphoshine)palladium(0) (1.2 g) in toluene (75 ml) and ethanol (5 ml) was stirred and heated at 80-90° C. for 4 hours. Complete consumption of SM (starting material), formation of product and some homocoupled product. The mixture was cooled to room temperature, water (100 ml) was added and the mixture stirred vigorously for 5 minutes. A clear two phase mixture was formed. The layers were separated and the aqueous phase washed with water (100 ml). The solvent was evaporated to leave a yellow-brown solid (11 g).

A further batch of crude product was prepared by as follows. A mixture of 2-(5-chloro-2-{[(4-chloro-2-fluorophenyl)methyl]oxy}phenyl)-4,4,5,5-tetramethyl-1,3,2-dioxaborolane (16 g), ethyl 6-(chloromethyl)-2-pyridinecarboxylate (8 g), K₂CO₃ (11.2 g) and Pd(PPh)₄ (tetrakis(triphenylphoshine)palladium(0), 2.4 g) in toluene (150 ml) and ethanol (10 ml) was stirred and heated at 80-90° C. for 6 hours. HPLC showed complete consumption of SM (starting material), formation of product and some homocoupled material. The mixture was cooled to room temperature, water (150 ml) was added and the mixture stirred vigorously for 5 minutes. A clear two phase mixture was formed. The layers were separated and the aqueous phase washed with water (150 ml). The solvent was evaporated to leave a yellow-brown solid (22 g).

The two batches were combined and dissolved in MDC (dichloromethane, 200 ml). The solution was filtered to remove a small amount of insoluble material. The solution was evaporated and the residue recrystallised from ethanol (170 ml) with hot filtration. The solution was cooled to room temperature for 2 hours, then 0-5° C. for 2 hours, then the solid product was filtered off, washed with ethanol (25 ml) and dried in a vacuum oven for 18 hours at 45° C. to give the title compound (21.2 g). HPLC showed some impurities.

### Reaction 2

Toluene (55 ml) and ethanol (55 ml) were added to a mixture of 2-(5-chloro-2-{[(4-chloro-2-fluorophenyl)methyl]oxy}phenyl)-4,4,5,5-tetramethyl-1,3,2-dioxaborolane (11 g, 27 mol), ethyl 6-(chloromethyl)-2-pyridinecarboxylate (5.5 g, 27 mol), K₂CO₃ (7.7 g, 54 mol) and (tetrakis(triphenylphoshine)palladium(0), (1.65 g, 5 mol %) and the mixture was heated at 80-90° C. for 1 hour. Additional toluene (55 ml) was added and the mixture was cooled to room temperature. Water (100 ml) was added and the mixture was stirred vigorously for 5 minutes. The layers were separated and the organic phase was washed with water. The solvent was evaporated to leave a brown semi-solid. The crude material was re-crystallised from ethanol (75 ml) with hot filtration. The filtrate was cooled to 0.5° C. for 2 hours. The product was filtered, washed with ethanol and dried in a vacuum oven at 50° C. overnight. A 7 g sample was purified by chromatography on silica gel (70 g), eluting with MDC (100 ml fractions taken). Fractions 2-14 were combined and evaporated to give a white solid, which was recrystallised from ethanol (25 ml).

### Step (f) Sodium 6-[(5-chloro-2-{[(4-chloro-2-fluorophenyl)methyl]oxy}phenyl)methyl]-2-pyridinecarboxylate

Ethyl 6-[(5-chloro-2-{[(4-chloro-2-fluorophenyl)methyl]oxy}phenyl)methyl]-2-pyridinecarboxylate (2 g) was dissolved in ethanol (15 ml) at reflux. 2M Sodium hydroxide (3.4 ml) was added and the solution heated under reflux for 30 minutes. No residual starting material by HPLC. The solution was filtered and the filter washed with a mixture of hot ethanol (5 ml) and water (5 ml). The combined filtrate and wash were re-heated to reflux, and water (15 ml) added dropwise over ˜5 minutes and the clear solution allowed to cool slowly to room temperature. The product crystallised rapidly at ˜35° C. The resulting thick suspension was cooled to 20-25° C. and stirred for 1 hour. The product was isolated and washed with 1:3 ethanol:water (20 ml) and then dried overnight at 50° C. in vacuo to give the title compound (1.94 g).

### Description 2

### 1-Chloro-4-[(2-methylpropyl)oxy]benzene (D2)

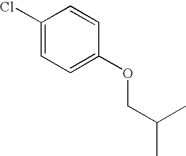

4-Chlorophenol (25 g, 0.194 mol), K₂CO₃ (32 g, 0.23 mol) and isobutyl bromide (21.5 mL, 0.214 mol) in DMF (150 mL) were heated at 90° C. overnight. More isobutyl bromide (10 mL) was added and the mixture was stirred for further 6 hours. The mixture was then cooled, diluted with water and extracted with EtOAc (×3). The combined organic phases were dried (MgSO₄) and evaporated to give the title compound.

¹HNMR (CDCl₃): δ 7.23-7.19 (2H, m), 6.83-6.79 (2H, m), 3.67 (2H, d, J=6.4), 2.09-2.03 (1H, m), 1.01 (6H, d, J=6.8).

### Description 3

### 4-Chloro-2-iodo-1-[(2-methylpropyl)oxy]benzene (D3)

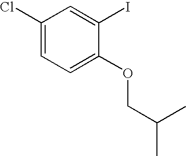

1-Chloro-4-[(2-methylpropyl)oxy]benzene (33.3 g, 0.18 mol; may be prepared as described in D2), iodine (23 g, 0.09 mol) and selectfluor (63.7 g, 0.18 mol) were stirred in dry acetonitrile (500 mL) at room temperature until the solution decolorized. The solvent was evaporated on a rotary evaporator keeping the bath temperature <30° C. The residue was portioned between diethyl ether and sodium thiosulphate solution; the organic phase was washed with water and brine, dried and evaporated to give the title compound as brown liquid.

¹HNMR (CDCl₃): δ 7.73 (1H, d, J=2.4), 7.24 (1H, dd, J=2.4, 8.8), 6.67 (1H, d, J=8.8), 3.73 (2H, d, J=6.4), 2.17-2.1 (1H, m), 1.07 (6H, d, J=6.8).

### Description 4

### {5-Chloro-2-[(2-methylpropyl)oxy]phenyl}boronic Acid (D4)

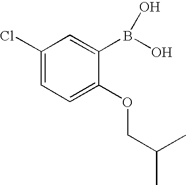

To solution of 4-chloro-2-iodo-1-[(2-methylpropyl)oxy]benzene (52 g, 0.166 mol; may be prepared as described in D3) in dry THF (400 mL) under argon, at −40° C., isopropyl magnesium chloride (2M in THF, 166 mL, 0.332 mol) was added dropwise over 40 min. The reaction mixture was stirred at −40° C. for other 30 min, then cooled to −78° C. Tiisopropyl borate (76.5 mL, 0.332 mol) was added dropwise over 30 min, after complete addition the mixture was stirred at −78° C. for other 30 min then allowed to reach room temperature. 2M HCl (400 mL) was added to the mixture, stirred at room temperature for 30 min, then aqueous layer was extracted with Et₂O(×2). The combined organic phases were dried and evaporated; the residue was triturated with hexane to give an off-white solid (13 g). The mother liquor was evaporated and chromatographed on a biotage using 15% of ethyl acetate in hexane to give a yellowish solid (4.7 g).

LCMS Rt=2.96, [MH] 226.3, 227.2

### Description 5

### Ethyl 6-({5-chloro-2-[(2-methylpropyl)oxy]phenyl}methyl)-2-pyridinecarboxylate (D5)

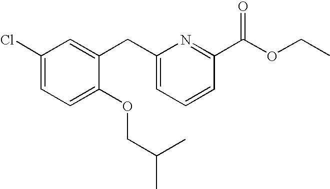

{5-Chloro-2-[(2-methylpropyl)oxy]phenyl}boronic acid (18 g, 78 mmol; may be prepared as described in D4), ethyl 6-(bromomethyl)-2-pyridinecarboxylate (15.6 g, 78 mmol), potassium carbonate (43.2 g, 312 mmol) and Pd(PPh₃)₄ (9 g, 0.78 mmol) were stirred in 1:1 toluene:ethanol (450 mL) under argon, at 90° C., for 3 hours. The mixture was then cooled, some of the solvent was evaporated; the residue was diluted with water and extracted with diethyl ether. The organic phase was dried and evaporated. The residue was purified by flash chromatography using 8% of ethyl acetate in hexane (9.3 g).

LCMS Rt=3.73, [MH⁺] 348.1, 350.1

### Description 6

### 6-({5-Chloro-2-[(2-methylpropyl)oxy]phenyl}methyl)-2-pyridinecarboxylic Acid (D6)

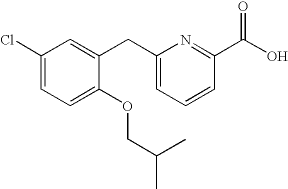

Ethyl 6-({5-chloro-2-[(2-methylpropyl)oxy]phenyl}methyl)-2-pyridinecarboxylate (10.8 g, 0.031 mol; may be prepared as described in D5) was dissolved in ethanol and NaOH 2M (25 mL) added. The reaction mixture was stirred at 50° C. for two hours. Solvent was evaporated, the residue was diluted with water, acidified with acetic acid and extracted with EtOAc (×2). The combined organic phases were dried, evaporated, azeotroped with toluene to give the title compound as a yellow gum (10.2 g).

LCMS Rt=2.97, [MH⁺] 320.2, [MH⁻] 318.2, 320.2

### Description 7

### 1-Chloro-4-[(phenylmethyl)oxy]benzene (D7)

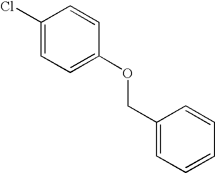

4-Chlorophenol (25 g, 0.194 mol), K₂CO₃ (32 g, 0.23 mol) and benzyl bromide (25.4 mL, 0.214 mol) in acetone (150 mL) were refluxed for 4 hours. The mixture was then cooled; the solid was filtered off and washed with more acetone. The solid was triturated with hexane to give the title compound as white solid (30.6 g).

LCMS Rt=3.45, [MH⁻] 217.3, 219.2

### Description 8

### 4-Chloro-2-iodo-1-[(phenylmethyl)oxy]benzene (D8)

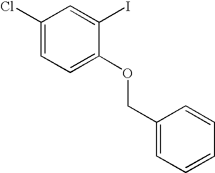

Prepared in a similar manner to D3. ¹HNMR (CDCl₃): δ 7.76 (1H, d, J=2.4), 7.47-7.21 (6H, m), 6.75 (1H, d, J=8.8), 5.13 (2H, s).

### Description 9

### {5-Chloro-2-[(phenylmethyl)oxy]phenyl}boronic Acid (D9)

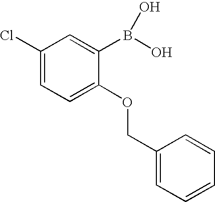

Prepared in a similar manner to D4.

¹HNMR (CDCl₃): δ 7.81 (1H, d, J=2.8), 7.44-7.35 (6H, m), 6.90 (1H, d, J=8.8), 5.8 (2H, s), 5.12 (2H, s).

### Description 10

### Ethyl 6-({5-chloro-2-[(phenylmethyl)oxy]phenyl}methyl)-2-pyridinecarboxylate (D10)

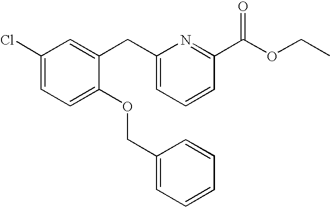

Prepared in a similar manner to D5,

LCMS Rt=3.63, [MH⁺] 1382.2, 385.2

### Description 11

### 6-({5-Chloro-2-[(phenylmethyl)oxy]phenyl}methyl)-2-pyridinecarboxylic Acid (D11)

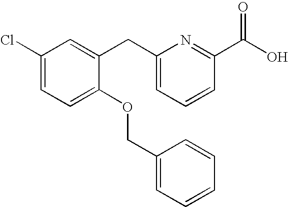

Ethyl 6-({5-chloro-2-[(phenylmethyl)oxy]phenyl}methyl)-2-pyridinecarboxylate (8.38 g, 22 mmol; may be prepared as described in D10) was dissolved in ethanol (95 mL) and NaOH 2M (35 mL) added. The reaction mixture was stirred at room temperature for 1 hour and 30 min. The solvent was evaporated, the residue was diluted with water, acidified with acetic acid and extracted with EtOAc (×3). The combined organic phases were dried (MgSO₄), evaporated, azeotroped with toluene to give the title compound (7.61 g)

LCMS Rt=2.95, [MH⁺] 354.1, 356.1, [MH⁻] 352.2, 354.2

### Description 12

### 6-({5-Chloro-2-[(phenylmethyl)oxy]phenyl}methyl)-2-pyridinecarbonitrile (D12)

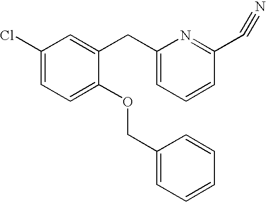

Prepared in a similar manner to D15.

LCMS Rt=3.61 [MH+] 335.1, 337.1, [MH⁻] 333.2

### Description 13

### 6-({5-Chloro-2-[(phenylmethyl)oxy]phenyl}methyl)-2-pyridinecarboxamide (D13)

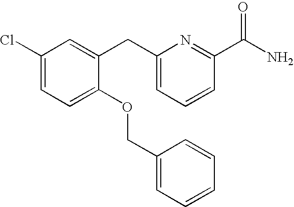

Prepared in a similar manner to E44.

LCMS Rt=3.15 [MH+] 353.4, 355.4

### Description 14

### Methyl 6-({5-chloro-2-[(phenylmethyl)oxy]phenyl}methyl)-2-pyridinecarboximidoate Hydrochloride (D14)

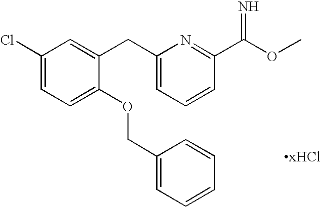

Prepared in a similar manner to D16 using 0.8 equivalents of sodium methoxide.

LCMS Rt=2.84 [MH⁺] 367.1, [MH⁻] 365.3

### Description 15

### 6-({5-Chloro-2-[(2-methylpropyl)oxy]phenyl}methyl)-2-pyridinecarbonitrile (D15)

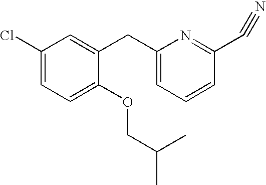

6-({5-Chloro-2-[(2-methylpropyl)oxy]phenyl}methyl)-2-pyridinecarboxamide (370 mg, 1.16 mmol, may be prepared as described in E41) was dissolved in 2 mL of phosphorus oxychloride and heated at 60° C. for 4 hours. The mixture was then cooled, poured onto ice and 2M NaOH added until basic pH, extracted with diethyl ether(×2). Combined organics were dried (MgSO₄) and evaporated to dryness. The residue was purified on an SPE silica cartridge using 15% of ethyl acetate in hexane to give a yellowish gum (300 mg).

LCMS Rt=3.63 [MH+] 301.2

### Description 16

### Methyl 6-({5-chloro-2-[(2-methylpropyl)oxy]phenyl}methyl)-2-pyridinecarboximidoate Hydrochloride (D16)

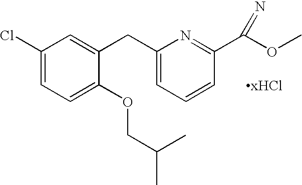

6-({5-Chloro-2-[(2-methylpropyl)oxy]phenyl}methyl)-2-pyridinecarbonitrile (300 mg, 0.99 mmol, may be prepared as described in D15) was dissolved in methanol (4 mL) and sodium methoxide (6 mg, 0.099 mmol) was added. The solution was stirred at room temperature until all the starting material disappeared (followed by LC/MS). The solvent was evaporated to give a pink oil (333 mg).

LCMS Rt=2.61 [MH+] 469.1, 471.1, [MH⁻] 467.2, 469.2

The hydrochloride salt was prepared dissolving the methyl 6-({5-chloro-2-[(2-methylpropyl)oxy]phenyl}methyl)-2-pyridinecarboximidoate in ethanol and treated with 1M HCl in diethyl ether, stirred for 5 minutes and evaporated.

### Description 17

### Methyl 4-({[6-({5-chloro-2-[(2-methylpropyl)oxy]phenyl}methyl)-2-pyridinyl]amino}carbonyl)benzoate (D17)

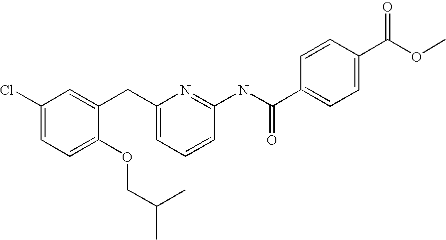

Oxalyl chloride (426 μL, 4.8 mmol) was added to a suspension of 4-[(methyloxy)carbonyl]benzoic acid (800 mg, 4.4 mmol) in DCM (20 mL) under argon followed by a drop of DMF. The mixture was stirred for 1 hour and evaporated to give a white solid that was added to a solution of 6-({5-chloro-2-[(2-methylpropyl)oxy]phenyl}methyl)-2-pyridinamine (700 mg, 0.24 mmol, may be prepared as described in D30) and TEA (0.4 mL, 2.9 mmol) in DCM (8 mL). The reaction mixture was stirred at room temperature for 3 hours, diluted with more dichloromethane and washed with water. Organic phase was dried and evaporated. The residue was purified on the Flash Master II using hexane containing a gradient of ethyl acetate (20-25%) to yield the title compound as a white solid (530 mg, Y=48%).

LCMS Rt=3.92 [MH⁺] 453.2, 455.2 [MH⁻] 451.1, 453.1

### Description 18

### Methyl 2-[6-({5-chloro-2-[(2-methylpropyl)oxy]phenyl}methyl)-2-pyridinyl]-1H-benzimidazole-5-carboxylate (D18)

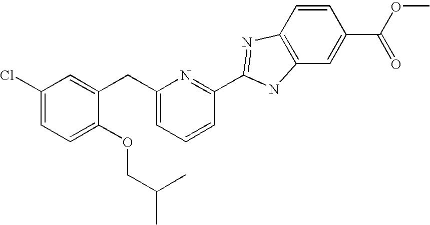

Methyl 6-({5-chloro-2-[(2-methylpropyl)oxy]phenyl}methyl)-2-pyridinecarboximidoate hydrochloride (1.6 g, 4.3 mmol, may be prepared as described in D16) was dissolved in ethanol (10 mL) and methyl 3,4-diaminobenzoate (719 mg, 4.3 mmol) added under argon. The reaction mixture was refluxed for 4 hours, cooled and evaporated. The crude was purified by reverse phase chromatography using a gradient of water and acetonitrile to give the title compound as yellow solid (610 mg).

LCMS Rt=3.83 [MH⁺] 450.2, 452.1 [MH⁻] 448.1, 450.3

### Description 19

### N-[6-({5-Chloro-2-[(2-methylpropyl)oxy]phenyl}methyl)-2-pyridinyl]-4-formylbenzamide (D19)

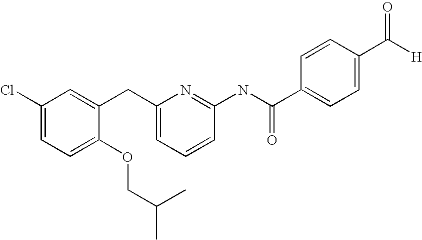

N-[6-({5-Chloro-2-[(2-methylpropyl)oxy]phenyl}methyl)-2-pyridinyl]-4-(hydroxymethyl)benzamide (450 mg, 1 mmol, may be prepared as described in E62) was dissolved in DCM (6 mL), Dess-Martin periodinane (451 mg, 1 mmol) was added to the mixture under argon. The reaction mixture was stirred for 1 hour, diluted with more DCM, washed with 10% sodium thiosulphate (10 mL) followed by saturated sodium bicarbonate solution (10 mL). The organic phase was dried (MgSO₄) and evaporated to give the title compound.

LCMS Rt=3.84 [MH⁺] 423.1, 425.1, 426.1 [MH⁻] 421.2, 423.2

### Description 20

### 6-[(5-Chloro-2-{[(4-chloro-2-fluorophenyl)methyl]oxy}phenyl)methyl]-N-(4-formylphenyl)-2-pyridinecarboxamide (D20)

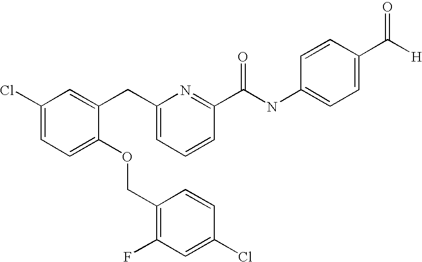

Prepared in a similar manner to D19.

LCMS Rt=4.02 [MH⁺] 509.2, 511.1

### Description 21

### Methyl 2-{6-[(5-chloro-2-{[(4-chloro-2-fluorophenyl)methyl]oxy}phenyl)methyl]-2-pyridinyl}-1H-benzimidazole-5-carboxylate (D21)

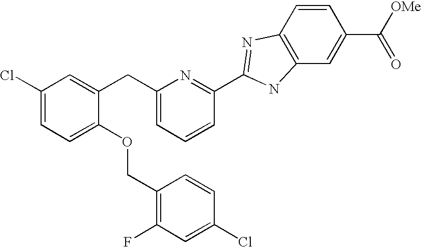

Prepared in a similar manner to D18.

LCMS Rt=3.86 [MH⁺] 536,539.1 [MH⁻] 534.1, 537.2

### Description 22

### {2-[6-({5-Chloro-2-[(2-methylpropyl)oxy]phenyl}methyl)-2-pyridinyl]-1H-benzimidazol-5-yl}methanol (D22)

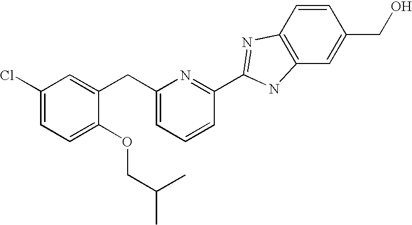

Methyl 2-[6-({5-chloro-2-[(2-methylpropyl)oxy]phenyl}methyl)-2-pyridinyl]-1H-benzimidazole-5-carboxylate (610 mg, 1.35 mmol, may be prepared as described in D18) was dissolved in 5 mL of THF under argon and cooled at −10° C. 1M LiAlH₄ in THF (1.49 mL, 1.49 mmol) was added and the solution was allowed to warm to room temperature. The dark mixture was quenched with water; the insoluble material that was formed was filtered off. The filtrate was then extracted with diethyl ether(×3), combined organics dried (MgSO₄) and evaporated to give the title compound (500 mg).

LCMS Rt=2.89 [MH⁺] 422.2, 425.1 [MH⁻] 420.3, 422.3

### Description 23

### 2-[6-({5-Chloro-2-[(2-methylpropyl)oxy]phenyl}methyl)-2-pyridinyl]-1H-benzimidazole-5-carbaldehyde (D23)

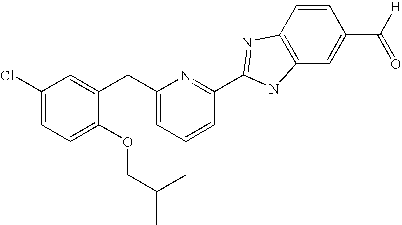

Prepared in a similar manner to D19.

LCMS Rt=3.7 [MH⁺] 420.2, 422.2 [MH⁻] 418.1, 420.1

### Description 24

### 2-{6-[(5-Chloro-2-{[(4-chloro-2-fluorophenyl)methyl]oxy}phenyl)methyl]-2-pyridinyl}-1H-benzimidazole-5-carbaldehyde (D24)

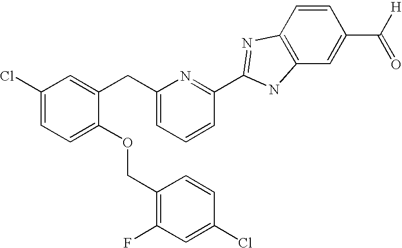

Prepared in a similar manner to D19.

LCMS Rt=3.75 [MH⁺] 506.2, 509.2 [MH⁻] 504, 507.9

### Description 25

### 6-[(5-Chloro-2-{[(4-chloro-2-fluorophenyl)methyl]oxy}phenyl)methyl]-2-pyridinecarbonitrile (D25)

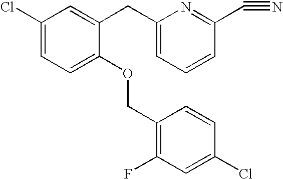

Prepared in a similar manner to D15.

LCMS Rt=3.81 [MH⁺] 387.1

### Description 26

### Methyl 6-[(5-chloro-2-{[(4-chloro-2-fluorophenyl)methyl]oxy}phenyl)methyl]-2-pyridinecarboximidoate Hydrochloride (D26)

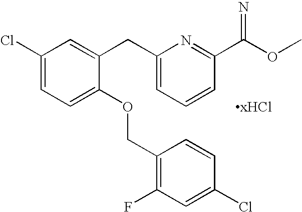

Prepared in a similar manner to D16.

LCMS Rt=3.11 [MH⁺] 419.1, 422.1

### Description 27

### (2-{6-[(5-Chloro-2-{[(4-chloro-2-fluorophenyl)methyl]oxy}phenyl)methyl]-2-pyridinyl}-1H-benzimidazol-5-yl)methanol (D27)

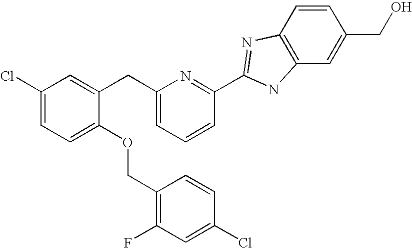

Prepared in a similar manner to D22 using 2.2 equivalent of LiAlH₄.

LCMS Rt=2.92 [MH⁺] 508, 510, 511, 512 [MH⁻] 506.1, 508.1, 509.1

### Description 28

### 1,1-Dimethylethyl [6-({5-chloro-2-[(2-methylpropyl)oxy]phenyl}methyl)-2-pyridinyl]carbamate (D28)

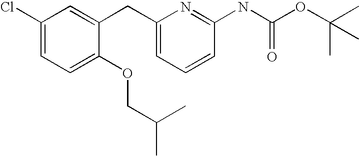

Prepared in a similar matter to E1.

LCMS Rt=4.09 [MH+] 391.

### Description 29

### 6-({5-Chloro-2-[(phenylmethyl)oxy]phenyl}methyl)-2-pyridinamine (D29)

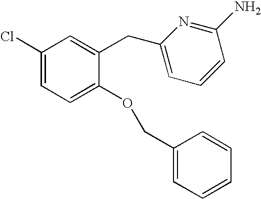

1,1-Dimethylethyl [6-({5-chloro-2-[(phenylmethyl)oxy]phenyl}methyl)-2-pyridinyl]carbamate (160 mg, 0.37 mmol; may be prepared as described in E1) was dissolved in 5 mL of 1:1 TFA:DCM and stirred at r.t. for 3 hours. The solvent was then evaporated and the residue dissolved in ethanol (5 mL) and 2M NaOH (3 mL), the resulting mixture was heated at 60° C. for 1 hour. The reaction was then allowed to cool to room temperature overnight. The solvent was evaporated and the residue was diluted with water, extracted with diethyl ether, dried (MgSO₄), filtered and evaporated to give the title compound as a yellowish solid (107 mg, Y=88%).

LCMS Rt=2.72 [MH+] 325.4, 327.4

### Description 30

### 6-({5-Chloro-2-[(2-methylpropyl)oxy]phenyl}methyl)-2-pyridinamine (D30)

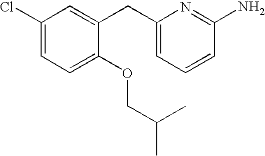

Prepared in a similar manner to D29.

LCMS Rt=2.13[MH+] 291.2, 294.2

### Example 1

### 1,1-Dimethylethyl [6-({5-chloro-2-[(phenylmethyl)oxy]phenyl}methyl)-2-pyridinyl]carbamate (E1)

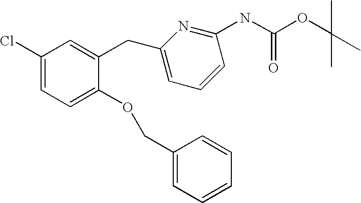

6-({5-Chloro-2-[(phenylmethyl)oxy]phenyl}methyl)-2-pyridinecarboxylic acid (3.7 g, 0.01 mol; may be prepared as described in D11), TEA (1.74 ml, 0.0125 mol) and diphenylphosphoryl azide (2.49 mL, 0.011 mol) in t-butanol (−100 mL) were refluxed for 6 hours. The mixture was then cooled, evaporated and the residue chromatographed on a pad of silica using 10% ethyl acetate/hexane mixture to yield the title compound (4.15 g).

LCMS Rt=4.3 [(MH-56)+] 369.4, 371.4

### Example 2

### N-[6-({5-Chloro-2-[(phenylmethyl)oxy]phenyl}methyl)-2-pyridinyl]-2-phenylacetamide (E2)

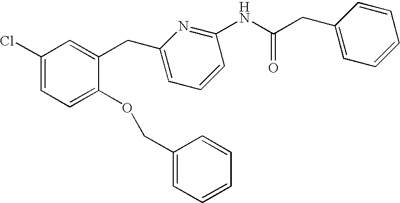

Phenyl acetyl chloride (36 μL, 0.27 mmol) was added to a mixture of 6-({5-chloro-2-[(phenylmethyl)oxy]phenyl}methyl)-2-pyridinamine (80 mg, 0.247 mmol; may be prepared as described in D29) and TEA (41 μL, 0.296 mmol) in dichloromethane (5 mL). The resulting mixture was stirred at room temperature overnight.

The reaction was diluted with ethyl acetate, washed with water, dried (MgSO₄) and evaporated.

Purification was carried out on an SPE column using hexane containing a gradient of ethyl acetate (10-20%) to yield the title compound as a white solid (84 mg, Y=77%).

LCMS Rt=4.09[MH+] 443.4, 445.4

### Examples 3-5

### E3-E5

The following compounds were prepared in a similar manner to E2.

| | | |
|---|---|---|
| 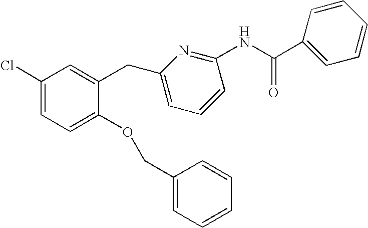 | N-[6-({5-Chloro-2-[(phenylmethyl)oxy]phenyl}methyl)-2-pyridinyl]benzamide (E3) | LCMS Rt =4.14[MH+] 429.4,431,4 |
| 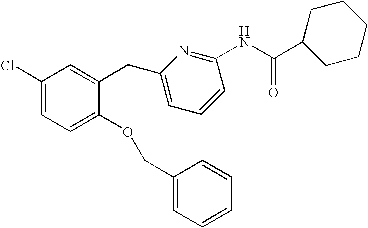 | N-[6-({5-Chloro-2-[(phenylmethyl)oxy]phenyl}methyl)-2-pyridinyl]cyclohexanecarboxamide (E4) | LCMS Rt =4.25[MH+] 435.4,437,4 |
| 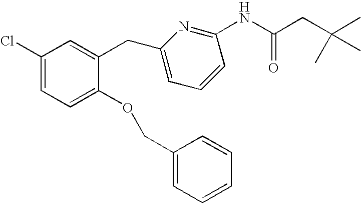 | N-[6-({5-Chloro-2-[(phenylmethyl)oxy]phenyl}methyl)-2-pyridinyl]-3,3-dimethylbutanamide (E5) | LCMS Rt = 4.18[MH+] 423.4,425,4 |

### Example 6

### 6-({5-Chloro-2-[(phenylmethyl)oxy]phenyl}methyl)-N-2-pyridinyl-2-pyridinecarboxamide (E6)

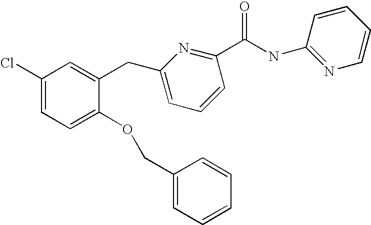

2-Aminopyridine (22 mg, 0.238 mmol) was added to a mixture of 6-({5-chloro-2-[(phenylmethyl)oxy]phenyl}methyl)-2-pyridinecarboxylic acid (70 mg, 0.198 mmol; may be prepared as described in D11), HOBt (32 mg, 0.238 mmol) and EDAC (45 mg, 0.238 mmol) in dichloromethane (4 mL). The reaction mixture was stirred at room temperature overnight, diluted with ethyl acetate and washed with sat. sodium bicarbonate solution and water. The organic phase was dried (Na₂SO₄), filtered, evaporated and the residue purified by flash chromatography.

LCMS Rt=4.21 [MH+] 430.1, 432.1

### Examples 7-12

### E7-E12

The following compounds were prepared in a similar manner to E6:

| | | |
|---|---|---|
| 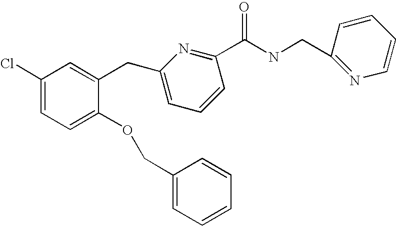 | 6-({5-Chloro-2-[(phenylmethyl)oxy]phenyl}methyl)-N-(2-pyridinylmethyl)-2-pyridinecarboxamide (E7) | LCMS Rt = 3.66[MH+]444.4, 446.4 |
| 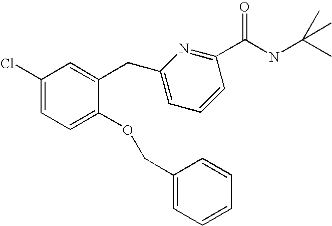 | 6-({5-Chloro-2-[(phenylmethyl)oxy]phenyl}methyl)-N-(1,1-dimethylethyl)-2-pyridinecarboxamide (E8) | LCMS Rt = 3.15[MH+]409.4, 411.5 |
| 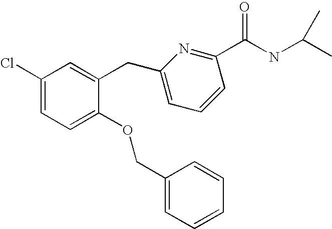 | 6-({5-Chloro-2-[(phenylmethyl)oxy]phenyl}methyl)-N-(1-methylethyl)-2-pyridinecarboxamide (E9) | LCMS Rt = 3.15[MH+]395.5, 397.4 |
| 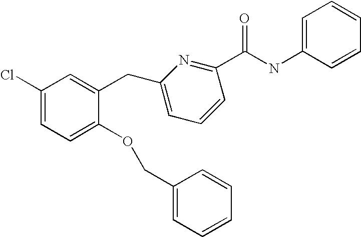 | 6-({5-Chloro-2-[(phenylmethyl)oxy]phenyl}methyl)-N-phenyl-2-pyridinecarboxamide(E10) | LCMS Rt = 4.31[MH+]429.1, 431.1 |
| 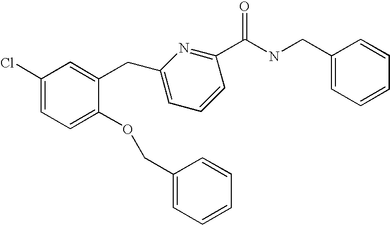 | 6-({5-Chloro-2-[(phenylmethyl)oxy]phenyl}methyl)-N-(phenylmethyl)-2-pyridinecarboxamide (E11) | LCMS Rt = 4.07[MH+]443.4, 445.4 |
| 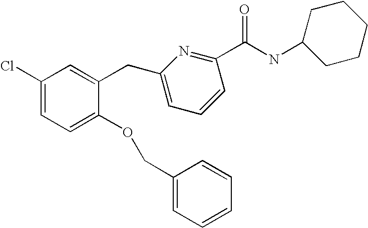 | 6-({5-Chloro-2-[(phenylmethyl)oxy]phenyl}methyl)-N-cyclohexyl-2-pyridinecarboxamide (E12) | LCMS Rt = 4.26[MH+]435.4, 437.4 |

### Example 13

### 6-[(5-Chloro-2-{[(4-chloro-2-fluorophenyl)methyl]oxy}phenyl)methyl]-N-[4-(hydroxymethyl)phenyl]-2-pyridinecarboxamide (E13)

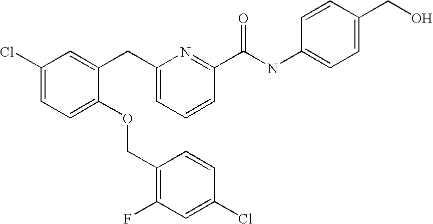

6-[(5-Chloro-2-{[(4-chloro-2-fluorophenyl)methyl]oxy}phenyl)methyl]-2-pyridinecarboxylic acid (2.86 g, 7.04 mmol, which may be prepared as described in D1) was dissolved in dichloromethane, 4-methylmorpholine (1.55 mL, 14.8 mmol), HOBt (1.14 g, 8.45 mmol) and EDAC (1.62 g, 8.45 mmol) were added. The reaction mixture was stirred at room temperature for 1 hour, the solvent was evaporated and ethyl acetate was added to the residue to give a suspension. The mixture was washed with saturated sodium bicarbonate solution (×2) and the solid precipitated was filtered off. The solid was dried and analyzed to confirm the title compound (2.37 g).

The organic layer was washed with 0.5M HCl, followed by brine and water; dried (MgSO₄) and evaporated to give more product as a solid (1.43 g).

LCMS Rt=3.69[MH+] 511.2, 513.2, 514.2

### Example 14

### N-[6-({5-Chloro-2-[(phenylmethyl)oxy]phenyl}methyl)-2-pyridinyl]tetrahydro-2H-pyran-4-carboxamide (E14)

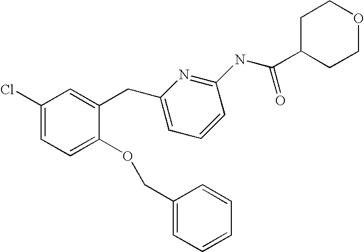

Tetrahydropyran-4-yl-carboxylic acid (35 mg, 0.271 mmol) was added to a mixture of 6-({5-chloro-2-[(phenylmethyl)oxy]phenyl}methyl)-2-pyridinamine (80 mg, 0.247 mmol, may be prepared as described in D29), EDAC (57 mg, 0.296 mmol) and HOBt (40 mg, 0.296 mmol) in dichloromethane (4 mL) and stirred at room temperature overnight. The reaction mixture was then evaporated and the residue chromatographed on a SPE silica column to give the title compound (35 mg).

LCMS Rt=4.68[MH+] 437.4, 439.5

### Examples 15-18

### E15-E18

The following compounds were prepared in a similar manner to E14.

| | | |
|---|---|---|
| 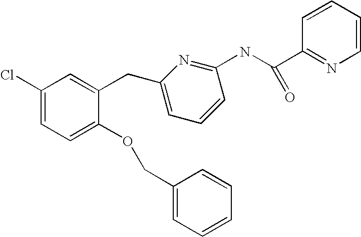 | N-[6-({5-Chloro-2-[(phenylmethyl)oxy]phenyl}methyl)-2-pyridinyl]-2-pyridinecarboxamide (E15) | LCMS Rt = 4.30[MH+]430.4, 432.4 |
| 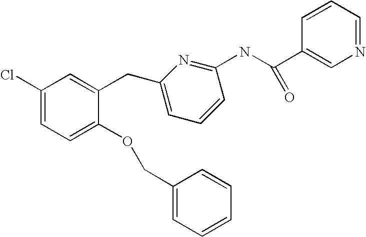 | N-[6-({5-Chloro-2-[(phenylmethyl)oxy]phenyl}methyl)-2-pyridinyl]-3-pyridinecarboxamide (E16) | LCMS Rt = 3.75[MH+]430.4, 432.4 |
| 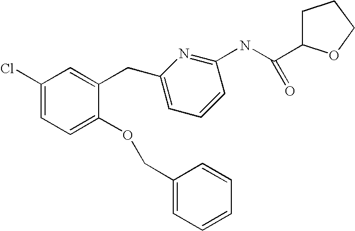 | N-[6-({5-Chloro-2-[(phenylmethyl)oxy]phenyl}methyl)-2-pyridinyl]tetrahydro-2-furancarboxamide (E17) | LCMS Rt = 4.85[MH+]423.4, 425.4 |
| 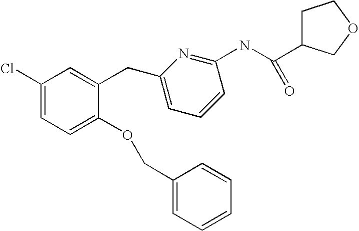 | N-[6-({5-Chloro-2-[(phenylmethyl)oxy]phenyl}methyl)-2-pyridinyl]tetrahydro-3-furancarboxamide (E18) | LCMS Rt = 3.66[MH+]423.4, 425.4 |

### Example 19

### 2-{6-[(5-Chloro-2-[([(4-chloro-2-fluorophenyl)methyl]oxy}phenyl)methyl]-2-pyridinyl]-1H-benzimidazole Hydrochloride (E19)

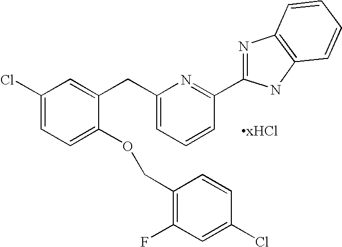

6-[(5-Chloro-2-{[(4-chloro-2-fluorophenyl)methyl]oxy}phenyl)methyl]-2-pyridinecarboxylic acid (150 mg, 0.37 mmol, may be prepared as described in D1) and 1,2-phenylenediamine (40 mg, 0.37 mmol) in POCl₃ were heated at 100° C. for 5 hours. The reaction mixture was cooled and poured onto ice and sat. sodium bicarbonate solution was added to pH8. The solution was extracted with ethyl acetate (×3), dried, filtered and evaporated.

The residue was purified by flash chromatography using 5% methanol in ethyl acetate, after evaporation of the solvent the residue was treated with 1M HCl in diethyl ether and evaporated again to give the title compound.

LCMS Rt=3.43[MH+] 478.1, 482.1, [MH⁻] 476.1, 478.2, 480.1

### Examples 20-25

### E20-E25

The following compounds were prepared in a similar manner to E19 and they were purified either by flash chromatography or by MDAP.

| | | |
|---|---|---|
| 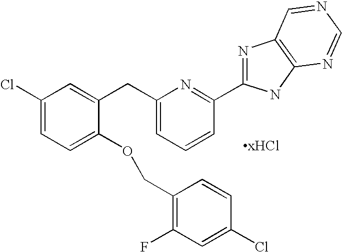 | 8-{6-[(5-Chloro-2-{[(4-chloro-2-fluorophenyl)methyl]oxy}phenyl)methyl]-2-pyridinyl}-1H-purinehydrochloride (E20) | LCMS Rt =3.37[MH+] 480.1,484.1 [MH⁻] 476.1,478.1, 481.3 |
| 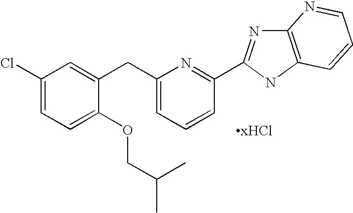 | 2-[6-({5-Chloro-2-[(2-methylpropyl)oxy]phenyl}methyl)-2-pyridinyl]-1H-imidazo[4,5-b]pyridine hydrochloride (E21) | LCMS Rt =3.34[MH+] 393.2,395.2 [MH⁻] 391.1,393.1 |
| 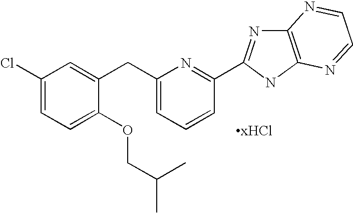 | 2-[6-({5-Chloro-2-[(2-methylpropyl)oxy]phenyl}methyl)-2-pyridinyl]-1H-imidazo[4,5-b]pyrazine hydrochloride (E22) | LCMS Rt =3.32[MH+] 394.1,396.1 [MH⁻] 392.1,394.1 |
| 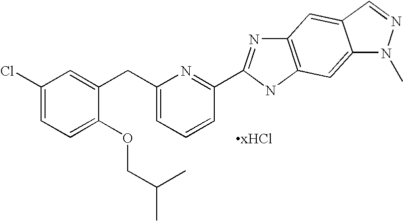 | 6-[6-({5-Chloro-2-[(2-methylpropyl)oxy]phenyl}methyl)-2-pyridinyl]-1-methyl-1,5-dihydroimidazo[4,5-f]indazolehydrochloride (E23) | LCMS Rt =3.29MH+] 446.1449.1 [MH⁻] 444.2,446.2 |
| 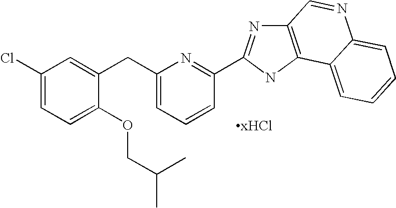 | 2-[6-({5-Chloro-2-[(2-methylpropyl)oxy]phenyl}methyl)-2-pyridinyl]-1H-imidazo[4,5-c]quinoline hydrochloride (E24) | LCMS Rt = 2.92MH+] 443.2, 445.2[MH⁻] 441.3, 443.3 |
| 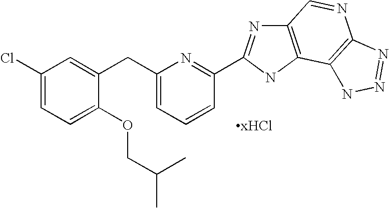 | 7-[6-({5-Chloro-2-[(2-methylpropyl)oxy]phenyl}methyl)-2-pyridinyl]-1,6-dihydroimidazo[4,5-e][1,2,3]benzotriazolehydrochloride (E25) | LCMS Rt = 3.23MH+] 433.1, 435.1[MH⁻] 431.2, 433.3 |

### Example 26

### 2-[6-({5-Chloro-2-[(phenylmethyl)oxy]phenyl}methyl)-2-pyridinyl]-5-(4-methyl-1-piperazinyl)-1H-benzimidazole Hydrochloride (E26)

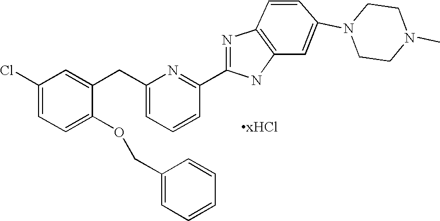

Methyl 6-({5-chloro-2-[(phenylmethyl)oxy]phenyl}methyl)-2-pyridinecarboximidoate hydrochloride (200 mg, 0.49 mmol, may be prepared as described in D14) was dissolved in ethanol (5 mL) and 4-(4-methyl-1-piperazinyl)-1,2-benzenediamine (100 mg, 0.49 mmol) added. The reaction mixture was refluxed for 5 hours, cooled and evaporated. The residue was diluted with NaOH 2M (4 mL) and extracted with diethyl ether (3×). Organics were dried (MgSO₄) and evaporated to dryness. The residue was purified on a MDAP; the product was treated with HCl 1M in diethyl ether (3 mL), stirred, concentrated in vacuo and triturated with diethyl ether to give a yellow solid.

LCMS Rt=2.17 [MH⁻] 522.3, 523.3

### Examples 27-28

### E27-E28

The following compounds were prepared in a similar manner to E26:

| | | |
|---|---|---|
| 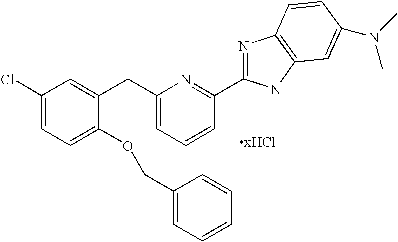 | 2-[6-({5-Chloro-2-[(phenylmethyl)oxy]phenyl}methyl)-2-pyridinyl]-N,N-dimethyl-1H-benzimidazol-5-aminehydrochloride (E27) | LCMS Rt = 2.61[MH+] 469.1, 471.1,[MH⁻] 467.2, 469.2 |
| 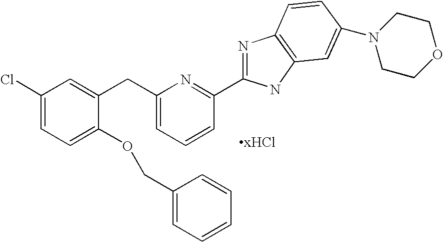 | 2-[6-({5-Chloro-2-[(phenylmethyl)oxy]phenyl}methyl)-2-pyridinyl]-5-(4-morpholinyl)-1H-benzimidazolehydrochloride (E28) | LCMS Rt = 2.85[MH+] 511.1, 413.1,[MH⁻] 509.2, 511.2 |

### Example 29

### 2-[6-({5-Chloro-2-[(phenylmethyl)oxy]phenyl}methyl)-2-pyridinyl]-5-methyl-1H-benzimidazole Hydrochloride (E29)

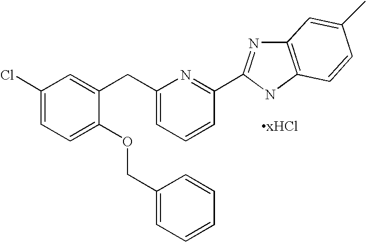

Methyl 6-({5-chloro-2-[(phenylmethyl)oxy]phenyl}methyl)-2-pyridinecarboximidoate hydrochloride (200 mg, assume 0.45 mmol, may be prepared as described in D14) was dissolved in ethanol (4 mL) and 4-methyl-1,2-benzenediamine (60 mg, 0.49 mmol) added. The reaction mixture was heated at 90° C. over the weekend, cooled and evaporated. The residue was purified on a SPE silica cartridge eluting with a mixture of hexane and ethyl acetate.

The white solid obtained was treated with HCl 1M in diethyl ether, stirred and concentrated in vacuo to give the hydrochloride salt.

LCMS Rt=3.19 [MH+] 440.1, 442.1, 443.1

### Examples 30-40

### E30-E40

The following compounds were prepared by a similar procedure used for E29, purification varied according to compounds:

| | | |
|---|---|---|
| 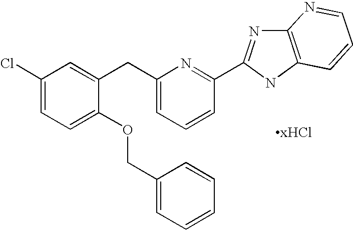 | 2-[6-({5-Chloro-2-[(phenylmethyl)oxy]phenyl}methyl)-2-pyridinyl]-1H-imidazo[4,5-b]pyridinehydrochloride (E30) | LCMS Rt = 3.25 [MH+]427.1, 429.1, 430., [MH⁻]425.2, 427.2 |
| 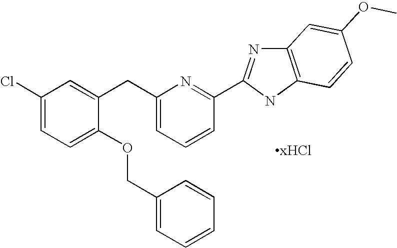 | 2-[6-({5-Chloro-2-[(phenylmethyl)oxy]phenyl}methyl)-2-pyridinyl]-5-(methyloxy)-1H-benzimidazolehydrochloride (E31) | LCMS Rt = 3.14 [MH+]456.1, 459.1[MH⁻] 454.2,456.2 |
| 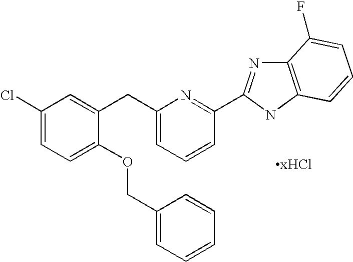 | 2-[6-({5-Chloro-2-[(phenylmethyl)oxy]phenyl}methyl)-2-pyridinyl]-4-fluoro-1H-benzimidazolehydrochioride (E32) | LCMS Rt = 3.77 [MH+]444.1, 446.1 [MH⁻] 442.2,444.2 |
| 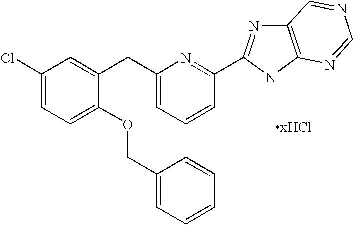 | 8-[6-({5-Chloro-2-[(phenylmethyl)oxy]phenyl}methyl)-2-pyridinyl]-1H-purinehydrochloride (E33) | LCMS Rt = 3.12 [MH+]428.1, 430 [MH⁻] 426.2,428.2 |
| 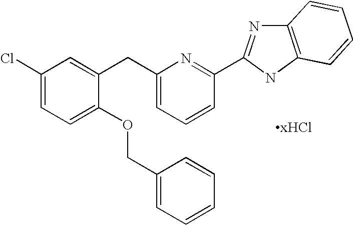 | 2-[6-({5-Chloro-2-[(phenylmethyl)oxy]phenyl}methyl)-2-pyridinyl]-1H-benzimidazole hydrochloride(E34) | LCMS Rt = 3.26 [MH+]426.2, 428.2 [MH⁻] 424.1,426.2 |
| 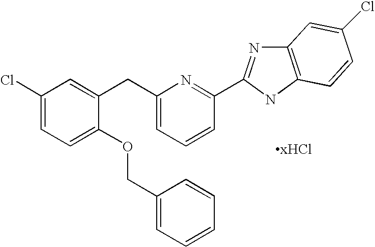 | 5-Chloro-2-[6-({5-Chloro-2-[(phenylmethyl)oxy]phenyl}methyl)-2-pyridinyl]-1H-benzimidazole hydrochloride(E35) | LCMS Rt = 3.8 [MH+]460.1 [MH⁻] 458.2 |
| 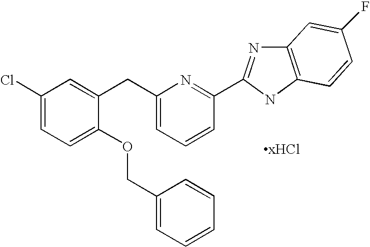 | 2-[6-({5-Chloro-2-[(phenylmethyl)oxy]phenyl}methyl)-2-pyridinyl]-5-fluoro-1H-benzimidazolehydrochloride (E36) | LCMS Rt = 3.69 [MH+]444.2, 446.2 [MH⁻] 442.1,444.1 |
| 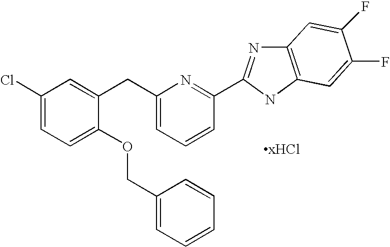 | 2-[6-({5-Chloro-2-[(phenylmethyl)oxy]phenyl}methyl)-2-pyridinyl]-5,6-difluoro-1H-benzimidazolehydrochloride (E37) | LCMS Rt = 3.8 [MH+]462.2, 464.2 [MH⁻] 460.1,462.1 |
| 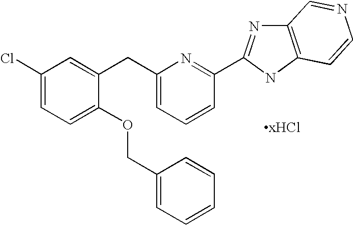 | 2-[6-({5-Chloro-2-[(phenylmethyl)oxy]phenyl}methyl)-2-pyridinyl]-1H-imidazo[4,5-c]pyridinehydrochloride (E38) | LCMS Rt = 2.41 [MH+]427.2, 429.2 [MH⁻] 425.1,427.1 |
| 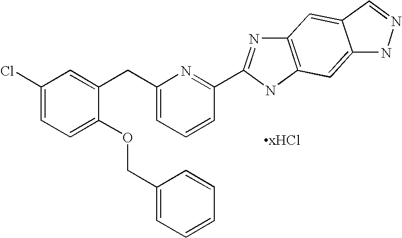 | 6-[6-({5-Chloro-2-[(phenylmethyl)oxy]phenyl}methyl)-2-pyridinyl]-1,5-dihydroimidazo[4,5-f]indazolehydrochloride (E39) | LCMS Rt = 2.89 [MH+]466.1, 468.1, 469.1[MH⁻]464.2, 466.2 |
| 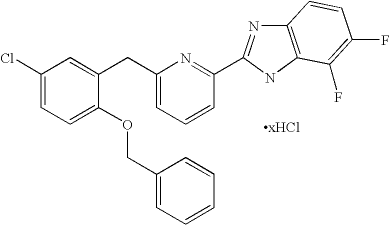 | 2-[6-({5-Chloro-2-[(phenylmethyl)oxy]phenyl}methyl)-2-pyridinyl]-4,5-difluoro-1H-benzimidazolehydrochloride (E40) | LCMS Rt = 3.88 [MH+]462.1, 464.1 [MH⁻] 460.2,462.1 |

### Example 41

### 6-({5-Chloro-2-[(2-methylpropyl)oxy]phenyl}methyl)-2-pyridinecarboxamide (E41)

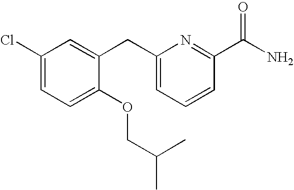

Oxalyl chloride (0.5 mL) was added to a suspension of 6-({5-chloro-2-[(2-methylpropyl)oxy]phenyl}methyl)-2-pyridinecarboxylic acid (415 mg, 1.29 mmol, may be prepared as described in D6) in 5 mL of DCM and one drop of DMF. The mixture was stirred at room temperature for 1 hour, solvent evaporated, dissolved in toluene and evaporated again. The brown oil obtained was dissolved in 15 mL of diethyl ether and 2.5 mL of aqueous ammonia (0.88) were added, stirred for 10 minutes; washed with water, 0.5M HCl and saturated sodium bicarbonate solution. The organic phase was then dried and evaporated to give a pale yellow solid (370 mg, Y=90%).

LCMS Rt=3.34 [MH+] 319.2, 322.2

### Example 42

### 2-({5-Chloro-2-[(2-methylpropyl)oxy]phenyl}methyl)-6-(1H-imidazol-2-yl)pyridine (E42)

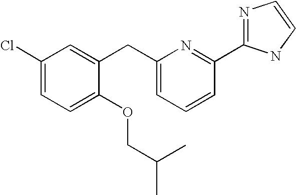

Methyl 6-({5-chloro-2-[(2-methylpropyl)oxy]phenyl}methyl)-2-pyridinecarboximidoate hydrochloride (150 mg, 0.45 mmol, may be prepared as described in D16) and 2,2-bis(methyloxy)ethanamine (63 μL, 0.59 mmol) in 3 mL of ethanol were refluxed overnight, evaporated and used without further purification. The residue was dissolved in a 1:1 mixture of 2M HCl and THF (3 mL in total). The solution was refluxed for 3 hours, cooled, diluted with ether and washed with 2M NaOH. The organic phase was dried, evaporated and purified on a MDAP.

LCMS Rt=2.38 [MH+] 342.4, 344.4

### Example 43

### 2-({5-Chloro-2-[(phenylmethyl)oxy]phenyl}methyl)-6-(1H-imidazol-2-yl)pyridine Hydrochloride (E43)

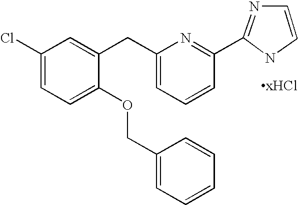

Prepared as described in E42. After the purification the compound was turned into the hydrochloride salt by treating with 1M HCl in diethyl ether and evaporated to give the title compound.

LCMS Rt=2.31 [MH+] 376.1, 379.

### Example 44

### 2-[6-({5-Chloro-2-[(2-methylpropyl)oxy]phenyl}methyl)-2-pyridinyl]-1H-benzimidazole (E44)

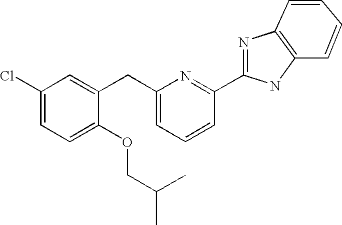

Prepared by a similar method used to E26, the title compound not treated with HCl to isolate the title compound as free base.

LCMS Rt=3.41 [MH+] 392.2, 394.2, [MH⁻] 390.3, 392.3, 393.3

### Example 45

### N-[6-({5-Chloro-2-[(2-methylpropyl)oxy]phenyl}methyl)-2-pyridinyl]-4-(4-morpholinylmethyl)benzamide Hydrochloride (E45)

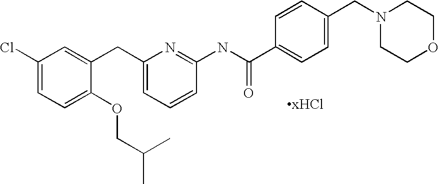

N-[6-({5-Chloro-2-[(2-methylpropyl)oxy]phenyl}methyl)-2-pyridinyl]-4-formylbenzamide (assumed 0.265 mmol, may be prepared as described in D19) was dissolved in 2 mL of DCM, acetic acid (15 μL, 0.265 mmol), sodium triacetoxy borohydride (56 mg, 0.265 mmol) and morpholine (23 μL, 0.265 mmol) were added. The reaction mixture was stirred under argon at room temperature overnight, diluted with more dichloromethane and washed with water. The organic layer was dried and evaporated. The residue was purified on a MDAP, followed by further purification on a FLEX, dissolved in methanol and treated with 1M HCl in diethyl ether (3 mL), stirred and evaporated to give a white solid (34 mg).

LCMS Rt=2.56 [MH⁺] 494.2 [MH⁻] 492.3, 494.2

### Examples 46-48

### E46-E48

Prepared in a similar manner to E45:

| | | |
|---|---|---|
| 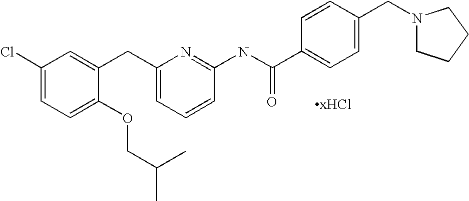 | N-[6-({5-Chloro-2-[(2-methylpropyl)oxy]phenyl}methyl)-2-pyridinyl]-4-(1-pyrrolidinylmethyl)benzamidehydrochloride (E46) | LCMS Rt =2.56 [MH⁺]478.2 [MH⁻]476.5, 478.2 |
| 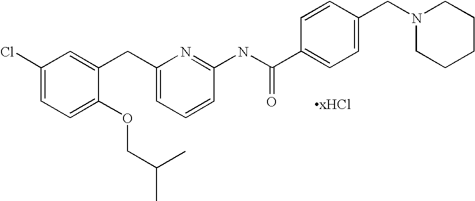 | N-[6-({5-Chloro-2-[(2-methylpropyl)oxy]phenyl}methyl)-2-pyridinyl]-4-(1-piperidinylmethyl)benzamidehydrochloride (E47) | LCMS Rt =2.61 [MH⁺]492.2, 495.2[MH⁻] 490.3,492.3 |
| 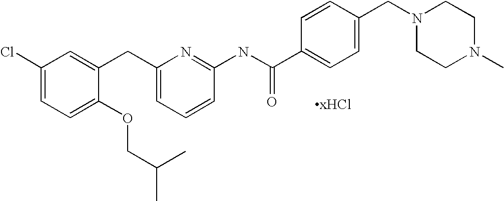 | N-[6-({5-Chloro-2-[(2-methylpropyl)oxy]phenyl)}methyl)-2-pyridinyl]-4-[(4-methyl-1-piperazinyl)methyl]benzamidehydrochloride (E48) | LCMS Rt =2.55 [MH⁺]507.2 [MH⁻]505.4, 506.2 |

### Examples 49-57

### E49-E57

### General Procedure 1

Sodium triacetoxyborohydride (79 mg, 0.37 mmol) or sodium borohydride (14 mg, 0.37 mmol) was added to a stirred solution of 2-[6-({5-chloro-2-[(2-methylpropyl)oxy]phenyl}methyl)-2-pyridinyl]-1H-benzimidazole-5-carbaldehyde (78 mg, 0.187 mmol, may be prepared as described in D23) and the appropriate amine (0.37 mmol) in THF (3 mL). The reaction mixture was stirred under argon at room temperature for 64 hours, diluted with ethyl acetate and washed with water. The organic phase was then dried, evaporated and purified on a silica column or on the MDAP, some products needed further purification on the FLEX.

The product obtained was dissolved in methanol and treated with 1M HCl in diethyl ether (2 mL) and evaporated to give the hydrochloride salt.

Following compounds were prepared using general procedure 1:

| | | |
|---|---|---|
| 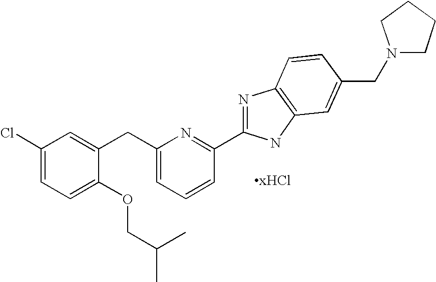 | 2-[6-({5-Chloro-2-[(2-methylpropyl)oxy]phenyl}methyl)-2-pyridinyl]-5-(1-pyrrolidinylmethyl)-1H-benzimidazole hydrochloride(E49) | LCMS Rt = 2.55[MH⁺] 475.2, [MH⁻]473.2, 474.1 |
| 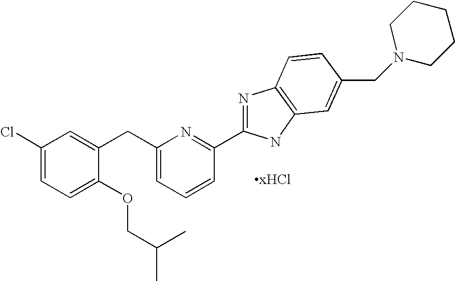 | 2-[6-({5-Chloro-2-[(2-methylpropyl)oxy]phenyl}methyl)-2-pyridinyl]-5-(1-piperidinylmethyl)-1H-benzimidazole hydrochloride(E50) | LCMS Rt = 2.68[MH⁺] 489.3, [MH⁻]487.2, 489.2, 490.1 |
| 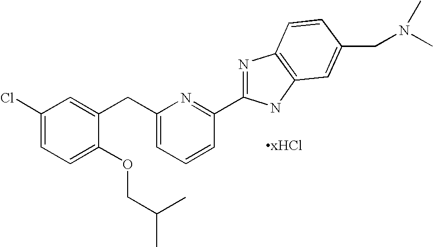 | ({2-[6-({5-Chloro-2-[(2-methylpropyl)oxy]phenyl}methyl)-2-pyridinyl]-1H-benzimidazol-5-yl}methyl)dimethylaminehydrochloride (E51) | LCMS Rt = 3.68[MH⁺] 449.3, 451.3[MH⁻] 447.2 |
| 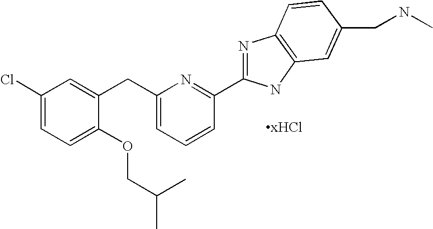 | ({2-[6-({5-Chloro-2-[(2-methylpropyl)oxy]phenyl}methyl)-2-pyridinyl]-1H-benzimidazol-5-yl}methyl)methylaminehydrochloride (E52) | LCMS Rt = 2.34[MH⁺] 435, [MH⁻]433.2, 435.1 |
| 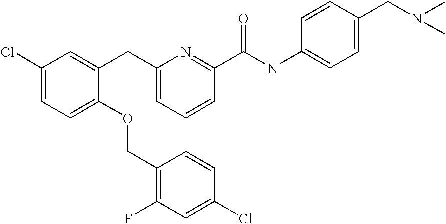 | 6-[(5-Chloro-2-{[(4-chloro-2-fluorophenyl)methy]oxy}phenyl)methyl]-N-{4-[(dimethylamino)methyl]phenyl}-2-pyridinecarboxamide (E53) | LCMS Rt = 2.68[MH⁻] 536.2, 537.1 |
| 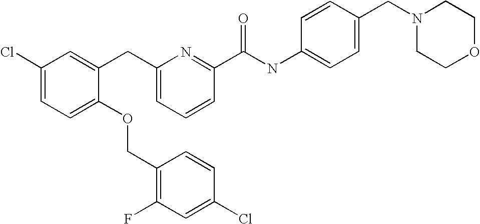 | 6-[(5-Chloro-2-{[(4-chloro-2-fluorophenyl)methyl]oxy}phenyl)methyl]-N-[4-(4-morpholinylmethyl)phenyl]-2-pyridinecarboxamide (E54) | LCMS Rt = 2.65[MH⁺] 580.2, [MH⁻]578.2 |
| 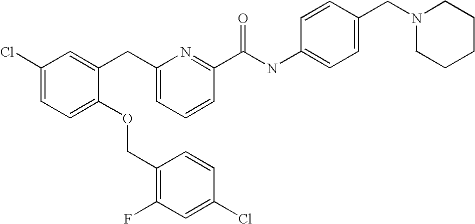 | 6-[(5-Chloro-2-{[(4-chloro-2-fluorophenyl)methyl]oxy}phenyl)methyl]-N-[4-(1-piperidinylmethyl)phenyl]-2-pyridinecarboxamide (E55) | LCMS Rt = 2.74[MH⁺] 578.1, 581.1,[MH^{-] 576.2} |
| 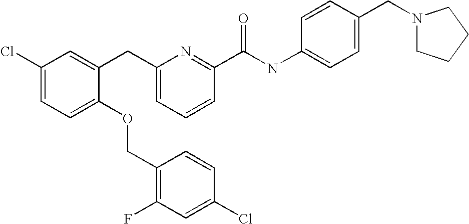 | 6-[(5-Chloro-2-{[(4-chloro-2-fluorophenyl)methyl]oxy}phenyl)methyl]-N-[4-(1-pyrrolidinylmethyl)phenyl]-2-pyridinecarboxamide (E56) | LCMS Rt = 2.71[MH⁺] 564.25, [MH⁻]562.25 |
| 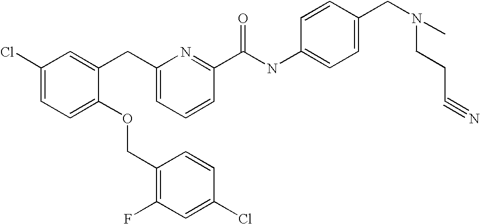 | 6-[(5-Chloro-2-{[(4-chloro-2-fluorophenyl)methyl]oxy}phenyl)methyl]-N-(4-{[(2-cyanoethyl)(methyl)amino]methyl}phenyl)-2-pyridinecarboxamide (E57) | LCMS Rt = 2.78[MH⁺] 576.2, [MH⁻]575.1 |

### Example 58

### [(2-{6-[(5-Chloro-2-{[(4-chloro-2-fluorophenyl)methyl]oxy}phenyl)methyl]-2-pyridinyl}-1H-benzimidazol-5-yl)methyl]dimethylamine Hydrochloride (E58)

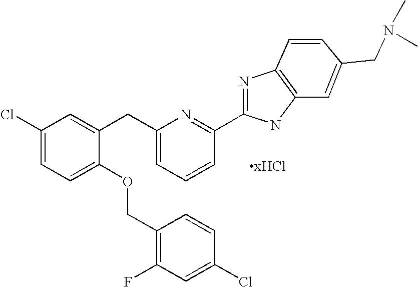

Sodium triacetoxyborohydride (50 mg, 0.237 mmol) was added to a stirred solution of 2-{6-[(5-chloro-2-{[(4-chloro-2-fluorophenyl)methyl]oxy}phenyl)methyl]-2-pyridinyl}-1H-benzimidazole-5-carbaldehyde (60 mg, 0.118 mmol, may be prepared as described in D24) and dimethylamine (42 μL, 0.237 mmol). The reaction mixture was stirred at room temperature overnight, sodium borohydride (10 mg) was added and the solution stirred for a further three hours. The mixture was then diluted with ethyl acetate and washed with water; the organic phase was dried, evaporated and purified on an SPE silica cartridge using 30% of methanol in dichloromethane. The white solid obtained was dissolved in methanol (4 mL) and treated with 1M HCl in diethyl ether (2 mL) and evaporated to give the title compound as a white solid (18 mg).

LCMS Rt=2.5 [MH⁻] 533.1, 536.09

### Example 59

### 1-{6-[(5-Chloro-2-{[(4-chloro-2-fluorophenyl)methyl]oxy}phenyl)methyl]-2-pyridinyl}-2,2,2-trifluoroethanol (E59)

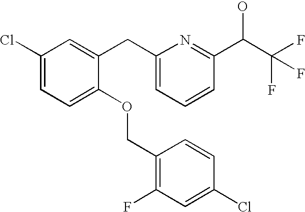

### Step (a) {6-[(5-Chloro-2-{[(4-chloro-2-fluorophenyl)methyl]oxy}phenyl)methyl]-2-pyridinyl}methanol

2M LiBH4 was added to ethyl 6-[(5-chloro-2-{[(4-chloro-2-fluorophenyl)methyl]oxy}phenyl)methyl]-2-pyridinecarboxylate (582.7 mg, 1.34 mmol, may be prepared as described in D1, Step (e)) in THF-EtOH (3.4 mL each, 0.2M) at r.t. then heated to reflux for 1 hour. Cooled to room temperature. Wet THF added slowly then EtOAc and 2M HCl. Layers separated and organic phase washed with sat. bicarb (saturated aqueous sodium bicarbonate solution), dried (Na₂SO₄), filtered and concentrated to give {6-[(5-chloro-2-{[(4-chloro-2-fluorophenyl)methyl]oxy}phenyl)methyl]-2-pyridinyl}methanol (537.6 mg, 100%) as a white foam.

LCMS Rt 2.77 min [ES+] 392.

### Step (b) 6-[(5-Chloro-2-{[(4-chloro-2-fluorophenyl)methyl]oxy}phenyl)methyl]-2-pyridinecarbaldehyde

Dess-Martin (D-M) periodinane (630 mg, 1.45 mmol) was added to a stirred solution of the alcohol, 6-[(5-chloro-2-{[(4-chloro-2-fluorophenyl)methyl]oxy}phenyl)methyl]-2-pyridinecarbaldehyde, (1.34 mmol) in DCM (6.7 mL) at r.t. Stirred for 2 hours. Excess oxidant destroyed by addition of a small volume of EtOH then dilution with DCM and washing with sat. bicarb. (saturated sodium bicarbonate solution) containing Na₂S₂O₃. DCM layer dried (Na₂SO₄), filtered and concentrated. Purified by chromatography on silica gel (20 g SPE) with hexane plus EtOAc (10-20%) to yield 6-[(5-chloro-2-{[(4-chloro-2-fluorophenyl)methyl]oxy}phenyl)methyl]-2-pyridinecarbaldehyde (226.8 mg, 51% for two steps).

LCMS Rt 3.80 [ES+] 390

### Step (c) 1-{6-[(5-Chloro-2-{[(4-chloro-2-fluorophenyl)methyl]oxy}phenyl)methyl]-2-pyridinyl}-2,2,2-trifluoroethanol

1M TBAF in THF (1.03 mL, 1.03 mmol) added dropwise to a solution of 6-[(5-chloro-2-{[(4-chloro-2-fluorophenyl)methyl]oxy}phenyl)methyl]-2-pyridinecarbaldehyde (266.8 mg, 0.68 mmol) and TMSCF₃ [trimethyl(trifluoromethyl)silane, 151 μL, 1.02 mmol] in THF at 0° C. Stirred for 2 days at room temperature then left to stand for 1 day. Diluted with Et₂O and washed with water, dried (Na₂SO₄), filtered and evaporated. Purified on 10 g SPE silica cartridge with hexane+EtOAc (5-10%) as eluent to give the title compound (148.8 mg, 47%).

LCMS Rt 3.77 [ES+] 460, 462, 464.

### Example 60

### 1,1-Dimethylethyl {6-[(5-chloro-2-{[(4-chloro-2-fluoronhenyl)methyl]oxy}phenyl)methyl]-2-pyridinyl}carbamate (E60)

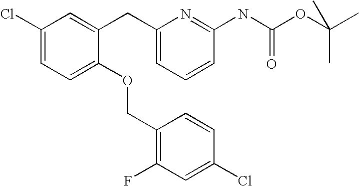

6-[(5-chloro-2-{[(4-chloro-2-fluorophenyl)methyl]oxy}phenyl)methyl]-2-pyridinecarboxylic acid (308.6 mg, 0.76 mmol, may be prepared as described in D1), t-BuOH (3 mL, 0.25M), DPPA (diphenylphosphoryl azide) (180 μL) and TEA (127 μL) were heated at reflux for 2.75 hours. Cooled to r.t., allowed to stand overnight. Diluted with EtOAc and washed with 2M HCl and sat. bicarb. (saturated sodium bicarbonate solution), dried (Na₂SO₄), filtered and concentrated.

### Example 61

### 1,1-Dimethylethyl {6-[(5-chloro-2-{[(4-chloro-2-fluorophenyl)methyl]oxy}phenyl)methyl]-2-pyridinyl}carbamate Hydrochloride (E61; Hydrochloride Salt of E60)

1,1-Dimethylethyl {6-[(5-chloro-2-{[(4-chloro-2-fluorophenyl)methyl]oxy}phenyl)methyl]-2-pyridinyl}carbamate (which may be prepared as described in E60) partially dissolved in THF (2 mL), 1M HCl in Et2O added (all dissolved) at r.t. Stirred at r.t. (precipitate formed). Evaporated. Et₂O added and decanted to leave the title compound (303.6 mg) as an off-white solid.

LCMS Rt 4.21 min [ES+] 421, 423.

### Example 62

### N-[6-({5-Chloro-2-[(2-methylpropyl)oxy]phenyl}methyl)-2-pyridinyl]-4-(hydroxymethyl)benzamide (E62)

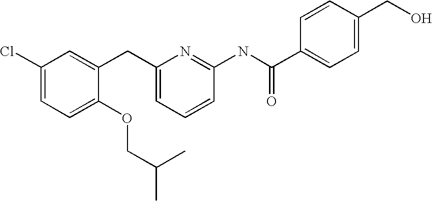

Methyl 4-({[6-({5-chloro-2-[(2-methylpropyl)oxy]phenyl}methyl)-2-pyridinyl]amino}carbonyl)benzoate (470 mg, 1 mmol, may be prepared as described in D17) was dissolved in 5 mL of THF under argon. 1M LiAlH₄ in THF (1.14 mL, 1.1 mmol) was added at −10° C., the mixture was then allowed to warm to room temperature, quenched with water and extracted with diethyl ether (×3). Combined organics were dried (MgSO₄) and evaporated to dryness to give the title compound as a white solid.

LCMS Rt=3.46 [MH⁺] 425.2, 427.1 [MH⁻] 423.1, 425.2

### Example 63

### 6-[(5-Chloro-2-{[(4-chloro-2-fluorophenyl)methyl]oxy}phenyl)methyl]-2-pyridinecarboxamide (E63)

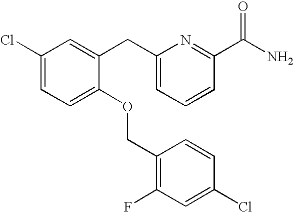

Prepared in a similar manner to E41 using the sodium salt instead of the free acid as starting material.

LCMS Rt=3.48 [MH⁺] 405.1, 408.1

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20mins and the pellet containing the membrane fraction is washed (optional) three times by suspension and centrifugation at 48,000×g for 20mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### Results

The compounds of examples 1-40 and 42-63 were tested in the binding assay for the human prostanoid EP₁ receptor. The results are expressed as pIC₅₀values. A pIC₅₀ is the negative logarithm₁₀ of the IC₅₀. The results given are averages of a number of experiments. The compounds of examples 1-40 and 42-63 had a pIC₅₀ value ≧6. More particularly, the compounds of examples 4-5, 13, 17-22, 27-31, 33-36, 39, 42-44, 51-53, 57-58 and 61-62 exhibited a pIC₅₀ value ≧7.

The compounds of examples 2-11, 16, 19-40, 43-52, 57, 58 and 60-63 were tested in the human EP₁ calcium mobilisation assay. The results are expressed as functional pKᵢ values. A functional pKᵢ is the negative logarithm₁₀ of the antagonist dissociation constant as determined in the human EP₁ calcium mobilisation assay. The results given are averages of a number of experiments. The compounds of examples 2-5, 7, 9, 13-14, 19-36, 38-39, 43-52, 57 and 60-63 exhibited a functional pKᵢ value ≧5.0. More particularly, the compounds of examples 3-5,7,9,14, 19-25, 28, 30-31, 33-36, 38-39, 43-45, 51-52 and 61 exhibited a functional pKᵢ value of ≧6.5. The compounds of examples 6, 8, 10, 11, 16, 37, 40 and 58 exhibited pIC₅₀ values of <5.

The compounds of examples 2-11, 13, 14, 16, 19-40, 43-52, 57, 58 and 60-63 were tested in the human EP₃ calcium mobilisation assay. The results are expressed as functional pKᵢ values. A functional pKᵢ is the negative logarithm₁₀ of the antagonist dissociation constant as determined in the human EP₃ calcium mobilisation assay. The results given are averages of a number of experiments. The compounds of examples 8-9, 14, 21, 23, 25, 33, 39, 43, 49 and 51 exhibited a functional pKi value of ≧5.5. The compounds of examples 9, 23, 25, 33 and 43 exhibited a functional pKᵢ value of ≧6. All other compounds tested were inactive, or exhibited a pKᵢ of <5.5.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 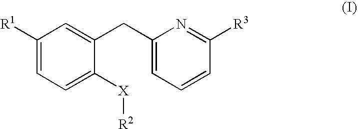
wherein:
R¹ is halogen;
X is oxygen or sulfur;
R² is isobutyl or optionally substituted benzyl;
R³ is selected from the group consisting of CO—NH—(CH₂)ₘ—R⁴, —NH—COO—R⁵, —NH—CO—(CH₂)ₙ—R⁶, and —C(H)(OH)—CF₃, or R³ represents optionally substituted imidazolyl, wherein the imidazole ring is optionally fused to give an optionally substituted bicyclic or tricyclic ring system;
R⁴ is selected from the group consisting of hydrogen, C₃₋₈ alkyl, C₃₋₈ cycloalkyl, optionally substituted phenyl and optionally substituted pyridyl;
R⁵ is t-butyl;
R⁶ is selected from the group consisting of C₃₋₈ alkyl, C₃₋₈ cycloalkyl, optionally substituted phenyl, optionally substituted pyridyl, tetrahydropyranyl and tetrahydrofuranyl;
m and n independently is 0 or 1;
and pharmaceutically acceptable salts thereof.

2. (canceled)

3. A pharmaceutical composition comprising a compound according to claim 1 or a pharmaceutically acceptable salt thereof together with a pharmaceutically acceptable excipient.

4. (canceled)

5. (canceled)

6. A method of treating a human or animal subject suffering from a condition which is mediated by the action of PGE₂ at EP₁ receptors which comprises administering to said subject an effective amount of a compound according to claim 1 or a pharmaceutically acceptable salt thereof.

7. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable salt thereof.

8. A method of treating a human or animal subject suffering from inflammatory pain, neuropathic pain or visceral pain which method comprises administering to said subject an effective amount of a compound according to claim 1 or a pharmaceutically acceptable salt thereof.

9-11. (canceled)
